(19) United States
(12) Patent Application Publication
(10) Pub. No.: US 2026/0085281 A1
(43) Pub. Date: Mar. 26, 2026

(54) Production of Culture Medium through Microbial Co-Hydrolysis of Plant Proteins

(71) Applicant: BTL Healthcare Technologies a.s., Prague (CZ)

(72) Inventors: Jiri Janousek, Jilove u Prahy (CZ); Sara Sediva, Praha 7 (CZ); Marketa Veresova, Praha 8 (CZ); Marek Popov, Praha 6 (CZ); Anezka Sigmundova, Libeznice (CZ)

(21) Appl. No.: 19/175,419

(22) Filed: Apr. 10, 2025

Related U.S. Application Data
(60) Provisional application No. 63/698,265, filed on Sep. 24, 2024.

(51) Int. Cl. C12N 5/00; C12P 21/06

(52) U.S. Cl. CPC C12N 5/0018; C12P 21/06; C12N 2501/998

(57) ABSTRACT

PROBLEM TO BE SOLVED: To solve the following problem: when waste seasoning is used in agricultural application for soil improvement and promotion of cultivation of vegetables, fruits and the like, using the waste seasoning as it is or after dilution does not provide soil improvement and a cultivation-promoting effect for vegetables, fruits and the like and, under certain circumstances, causes inhibition of cultivation; SOLUTION: In a seasoning waste treatment liquid and its production method, effective microorganisms essentially containing bacteria and fungi is added to an aqueous solution of the waste seasoning to culture the mixture under an anaerobic environment; COPYRIGHT: (C)2009,JPO&INPIT

### CROSS-REFERENCE

This application claims the benefit of U.S. Provisional Application No. 63/698,265, filed Sep. 24, 2024, the disclosure of which is incorporated by reference herein in its entirety.

The PCT application PCT/IB2024/053805 filed 18 Apr. 2024, and PCT application PCT/IB2024/059990 filed Oct. 11, 2024, and U.S. Provisional Patent Application No. 63/589,661 filed Oct. 12, 2023, and U.S. Provisional Patent Application No. 63/555,543 filed Feb. 20, 2024, and U.S. Provisional Patent Application No. 63/570,973 filed Mar. 28, 2024, and U.S. Provisional Patent Application No. 63/654,493 filed May 31, 2024, and U.S. Non-Provisional patent application Ser. No. 18/731,896 filed on Jun. 3, 2024, and U.S. Non-Provisional patent application Ser. No. 18/763,199 filed on Jul. 3, 2024, and U.S. Provisional Patent Application No. 63/698,265 filed Sep. 24, 2024. U.S. Provisional Patent Application No. 63/497,051 filed Apr. 19 2023. U.S. Provisional Patent Application No. 63/737,932 filed Dec. 23, 2024. U.S. Provisional Patent Application No. 63/760,147. U.S. Non-provisional patent application Ser. No. 18/927,171 filed Oct. 25, 2024. U.S. Non-provisional patent application Ser. No. 19/028,830 filed Jan. 17, 2025. U.S. Non-provisional patent application Ser. No. 19/028,930 filed Jan. 17, 2025. U.S. Non-provisional patent application Ser. No. 19/029,060 filed Jan. 17, 2025. U.S. Provisional Patent Application No. 63/765,920. U.S. Provisional Application No. 63/785,064 filed Apr. 8, 2025. U.S. Provisional Application No. 63/785,075 filed Apr. 8, 2025, and U.S. Provisional Application No. 63/786,408 filed Apr. 10, 2025 are all incorporated herein by reference in their entirety.

### FIELD OF THE INVENTION

The present invention is related to the field of cell cultures. In particular, it relates to systems and methods for production of culture medium for cell cultivation.

### BACKGROUND OF THE INVENTION

The global demand for sustainable and ethical alternatives to animal-derived food products has driven significant advancements in cellular agriculture, particularly in the production of cultured meat. This technology involves cultivating metazoan cells in controlled environments using nutrient-rich culture media that provide amino acids, growth factors, and essential nutrients required for cell proliferation and tissue formation.

A key challenge in this area is the formulation of animal-free culture media that are both nutritionally adequate and economically viable at an industrial scale. One promising approach is the use of plant-based protein hydrolysates, such as those derived from soy or pea protein, which can serve as a cost-effective and ethical source of amino acids and peptides.

However, plant-derived protein sources often present significant drawbacks. These include the presence of anti-nutritional compounds, most notably inositol hexaphosphate (phytic acid) and its derivatives. Such compounds are known to chelate essential minerals, leading to the formation of precipitates in the culture medium. Furthermore, they can interfere with cell growth and metabolic activity, compromising the reproducibility and efficiency of cell culture processes. In addition, protein hydrolysates prepared from plant-based sources may exhibit batch-to-batch variability, incomplete hydrolysis, and other biochemical inconsistencies that undermine their utility in sensitive biotechnological applications.

To address these challenges, the present invention introduces a novel and synergistic method for producing high-quality protein hydrolysates. This method involves the co-hydrolysis of two biologically distinct protein sources: (i) a plant-based protein isolate that is rich in protein but may contain undesirable compounds such as inositol hexaphosphate; and (ii) a microorganism, such as Saccharomyces cerevisiae, which not only contributes valuable proteins but also provides enzymes with specific activity during processing.

Particular microorganisms are known to produce enzymes with specific activity (e.g. proteolytic activity and phytase activity), particularly during autolysis or metabolic activity. These enzymes can degrade complex protein structures and cleave phosphate groups from inositol hexaphosphate and/or its derivatives. When the particular microorganism is combined with the plant-based protein source under appropriate processing conditions, there are benefits from both exogenously added enzymes and endogenously produced enzymes. This combination results in a cooperative hydrolysis mechanism in which the total amount of exogenously supplied enzymes can be significantly reduced. Moreover, particular microorganisms are capable of undergoing autolysis through various endogenously produced enzymes, thereby providing an additional source of protein beyond the plant-based protein source.

The synergistic action of endogenously produced enzymes by microorganisms and exogenously supplied enzymes results in more efficient breakdown of proteins into bioavailable peptides and amino acids while also removing inhibitory compounds such as inositol hexaphosphate. In parallel, the release of phosphate ions from inositol hexaphosphate and its derivatives degradation can help meet the phosphate requirements of cell culture media, potentially reducing or eliminating the need for additional inorganic phosphate supplementation.

Following enzymatic treatment, the resulting protein hydrolysate is typically subjected to sterilization and filtration steps. These are necessary to remove insoluble residues and microbial contaminants, yielding a purified, non-toxic, and cell-compatible protein hydrolysate. The final product may then be combined with other defined components to formulate complete, animal-free media suitable for supporting the growth of various metazoan cells, including those used for cultured meat production, tissue engineering, and recombinant protein production.

This co-hydrolysis approach enables a cost-effective, scalable, and nutritionally rich solution for next-generation culture media, overcoming limitations of single-source hydrolysates while reducing reliance on high-load enzyme supplementation and synthetic additives.

### BRIEF SUMMARY OF THE INVENTION

The present intention relates to the field of culture medium for cell cultures, comprising a plant-based protein material and yeast biomass. Natural production of phytases and proteases by yeasts acts synergistically with exogenously added phytase and proteases to enhance the enzymatic hydrolysis of the plant-based protein source. This results in the generation of a high-quality protein hydrolysate, providing a highly functional, culture medium suitable for supporting the growth and maintenance of non-human metazoan cells.

### BRIEF DESCRIPTION OF THE DRAWINGS

FIG. 1: illustrates scheme diagram of protein hydrolysate processing.

FIG. 2: illustrates the scheme of cultivation system used for cultivation of the non-human metazoan metazoan cells.

### DETAILED DESCRIPTION OF THE INVENTION

Disclosed herein are the methods for the production of a culture medium designed for the cultivation of non-human metazoan cells. The culture medium may comprise a source of protein with a nutritional function for the cells, signaling molecules, minerals, organic amines, saccharides, shear protectants, antifoaming agents, and vitamins. The source of protein, also referred to as substrate, source of protein or source of amino acids, may comprise protein hydrolysate prepared from soy, pea, rice, wheat, wheat gluten, corn, faba beans, alfalfa, hemp, chickpea, potato, pumpkin, rapeseed, red lentil, Spirulina, Chlorella, sunflower, water lentil, mung beans, flax, baker's yeast, brewer spent grain, distillers spent grain (DDGS), tomato pomace, in form of powder, lysate, concentrate, isolate, liquid, solid or any other appropriate form. The plant sources of protein from mentioned examples often contain phytin, phytic acid, inositol hexaphosphate, or phytate and/or any related form of those compounds, which will be referred to as inositol hexaphosphate and its derivatives in this document.

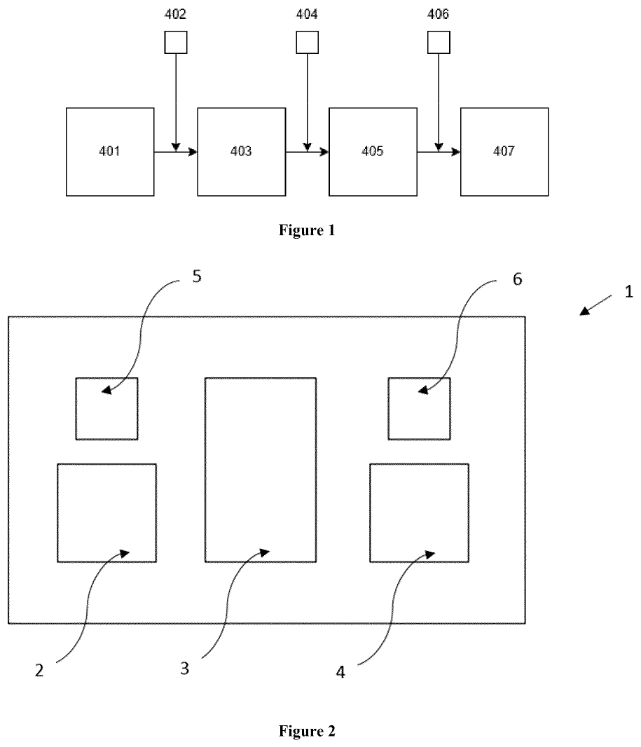

The protein hydrolysate processing depicted in FIG. 1 may comprise the following steps:
proteolysis of source of protein (401) by proteolytic enzymes (402) to result in formation of protein hydrolysate (403);
removal of inositol hexaphosphate and/or its derivatives from protein hydrolysate (403) by addition of enzymes having phytase activity and/or precipitating agent (404) to result in formation of modified protein hydrolysate (405);
removal of solid residues (406) from modified protein hydrolysate to result in formation of purified protein hydrolysate (407).

According to some aspects of the invention, the terms “source of amino acids”, “substrate”, “proteinous substrate”, “source of protein”, “protein source” may be interchangeable when referring to a component for the production of protein hydrolysate and/or the culture medium.

The term “derivatives” refers to molecules derived from inositol hexaphosphate, including inositol pentaphosphate, inositol tetraphosphate, inositol triphosphate, inositol diphosphate, inositol monophosphate intermediates, inositol or a combination thereof, as well as their combination with other substances, including ions or any other compounds with which these derivatives may commonly react.

Inositol phosphates may react with cations and form precipitates, which may lead to the blockage of filters within the cultivation process. Additionally, such precipitation may result in an imbalanced ionic concentration, thereby disrupting the optimal conditions necessary for cell growth.

The patent application discloses four aspects for removing inositol hexaphosphate and its derivatives from protein hydrolysate. The first aspect uses enzymes having phytase activity to degrade inositol hexaphosphate and its derivatives. The second aspect employs precipitating agents to form precipitates that can be separated from the culture medium. The third aspect combines both enzymatic removal and precipitation. The fourth aspect uses yeast biomass for the production of enzymes having phytase activity to degrade inositol hexaphosphate and its derivatives present in the plant-based protein source.

The source of protein for hydrolysis may be selected from an industrially scalable source of protein. Industrially scalable sources of amino acids include phototrophic organisms, such as land plants, green algae, red algae, brown algae, or other phototrophic eukaryotes, phototrophic prokaryotes such as cyanobacteria, or cultivated heterotrophic prokaryotes or eukaryotes, such as bacteria or yeast, or cultivated chemoautotrophic prokaryotes, such as hydrogen-oxidizing bacteria. The organism used as a source of protein may be able to synthesize all proteinogenic amino acids from inorganic nitrogen sources, such as ammonia ions, nitrate ions or molecular nitrogen. The hydrolysis may be performed, for example, in a hydrolysis tank, or in any other suitable device.

The hydrolysis may be performed on a protein isolate or concentrate or protein flour or protein meal or seed or grain from the source of protein, or on the whole biomass of the source of protein. The source of protein may be physically, mechanically or chemically pretreated, or steeped in water to induce germination, or subjected to methods such as soaking, blanching, removal of hull, husk or any other outer layer of the source of protein, milling, heat treatment or any other appropriate method to enhance the speed and efficiency of the hydrolysis process and to reduce the presence of antinutritional compounds. Saccharides, fats or other compounds may be removed from the biomass of the source of protein to facilitate easier processing. Examples of suitable industrially scalable sources of amino acids may include soy, pea, rice, wheat, wheat gluten, corn, faba beans, alfalfa, hemp, chickpea, potato, pumpkin, rapeseed, red lentil, Spirulina, Chlorella, sunflower, water lentil, mung beans, flax, brewer spent grain, distillers spent grain (DDGS), tomato pomace, in form of powder, lysate, concentrate, isolate, liquid, solid or any other appropriate form. The present invention is not limited to the listed exemplary sources of amino acids.

In one aspect of the invention, the incubation of the source of proteins in water to induce germination, or its pretreatment with soaking, blanching, milling, heat treatment or any other appropriate method may be performed in specific incubation time, wherein the incubation time may differ according to used technique.

The process of hydrolysis entails cleaving the original protein molecule into shorter peptide chains and/or single amino acids. As used herein, the term “protein hydrolysate” is understood to be a mix of amino acids, peptides and other molecules prepared from a suitable source of protein by any suitable method, including acidic, basic, or enzymatic hydrolysis, autolysis, thermal hydrolysis, or lysis by fermentation with a suitable microorganism which is able to break down the protein. The “protein hydrolysate” according to the present disclosure may be, for example, plant protein enzymatic hydrolysates, various types of yeast extracts or lysates (such as whole yeast autolysate), or algae acidic hydrolysate.

In one aspect of the invention, the microorganism used for fermentation may be from the group of Saccaromyces cerevisiae, Pichia pastoris, Schizosaccharomyces pombe, Candida utilis, Debaryomyces hansenii, or any other appropriate yeast.

Methods of protein hydrolysis may include acidic hydrolysis, basic hydrolysis, enzymatic hydrolysis, thermal hydrolysis or autolysis. Acidic hydrolysis subjects the source of protein to a very low pH, usually at an elevated temperature. The duration of reaction may be in a range of 1 hour to 96 hours, in a range of 2 hours to 72 hours, in a range of 4 hours to 48 hours, in a range of 4 hours to 36 hours, in a range of 4 hours to 24 hours or in a range of 4 hours to 12 hours. Acidic hydrolysis unfortunately leads to significant degradation of several amino acids, most notably tryptophan, which would then have to be sourced separately at significant costs. Significant degradation of some amino acids also occurs during basic hydrolysis, which subjects the source of protein to a very high pH, usually at an elevated temperature. Additionally, the acid or base used for the hydrolysis would have to be removed from the protein hydrolysate before it could be used to cultivate cells, presenting further complications. For example, when acidic hydrolysis is performed using hydrochloric acid, the acid may be removed by neutralization or evaporation. However, both processes are economically unfavorable because: i) neutralization process results in unfavorably high concentration of salts, which also need to be removed, and ii) evaporation is energy-intensive and the resulting HCl vapors pose a health and environmental hazard that would need to be solved. Thermal hydrolysis subjects the source of protein to very high temperatures at which the peptide bonds in the protein will break. However, at these temperatures, undesirable chemical reactions may occur—for example, some amino acids may break down or react with other compounds in the hydrolysate, for example through Maillard reactions with saccharides; additionally, thermal degradation of the reaction substrate may produce compounds harmful to the cultivated cells. The process of autolysis relies on the activity of the endogenous enzymes of the source organism to break down the source of protein, and this process is usually not very efficient and does not generally result in sufficient hydrolysis of the source protein. Additionally, proteins can be broken down by fermentation with organisms such as Bacillus licheniformis, Saccharomyces cerevisiae or Aspergillus oryzae, which produce a large amount of proteolytic enzymes. However, with this approach, some of the amino acids from the source of protein may be consumed by the organism that was used to break down the protein during the process of fermentation. Also, metabolic waste products and other compounds from the fermenting organism may contaminate the resulting lysate and adversely affect its properties in respect to mammalian cell cultivation.

The protein hydrolysate according to the invention may be obtained by enzymatic hydrolysis of a suitable source of protein. An industrially scalable source of protein is advantageous. In one aspect of the invention, soy protein isolate may be used as the source of protein for enzymatic hydrolysis. Advantageously, soy protein isolate has a favorable ratio of most amino acids for the purpose of mammalian cell cultivation. However, to achieve optimal levels of certain amino acids that may be present in lower concentrations, these amino acids may need to be supplemented separately in the culture media.

The source of protein for hydrolysis in solvent may be subjected to an initial thermal pretreatment to improve solubility and susceptibility to hydrolysis. The temperature during the thermal pretreatment may be in the range of 75° C. to 95° C., or in the range of 80° C. to 92.5° C., or in the range of 85° C. to 90° C. for a time in the range of 5 minutes to 120 minutes, in the range of 15 minutes to 60 minutes, or in the range of 30 minutes to 45 minutes.

In another aspect of the invention, the source of protein for hydrolysis in solvent may be subjected to an initial thermal pretreatment to improve solubility and susceptibility to hydrolysis. The temperature during the thermal pretreatment may be in the range of 70° C. to 99° C., or in the range of 80° C. to 98° C. or in the range of 85° C. to 97° C. for a time in the range of 5 minutes to 120 minutes, in the range of 15 minutes to 60 minutes, or in the range of 30 minutes to 45 minutes. In another aspect of the invention, the hydrolysis tank may be pressured, therefore temperatures above 100° C. might be used as well.

The concentration of the endoprotease, for example Alcalase, may be in the range of 0.01% to 10%, or in the range of 0.05% to 5%, or in the range of 0.1% to 1% expressed as a ratio of the concentration of enzyme to the concentration of protein in the reaction mixture. The concentration of the enzyme may be determined by the Bradford assay, BCA assay or other protein determination assays. The resulting solution has a basic to neutral pH, allowing for a high activity of Alcalase.

In some aspects of the invention, the endopeptidase enzyme may have endopeptidase activity in the range of 0.0001 to 300,000 TU, or in the range of 0.001 to 30,000 TU, or in the range of 0.01 to 3,000 TU per 1 g of enzyme preparation, wherein the one tyrosine unit (TU) is equal to amount of enzyme which liberates one micromole of tyrosine equivalents from casein at 40° C. and at pH 7. In other aspect of the invention, the endopeptidase enzyme may have endopeptidase activity in the range of 0.00001 to 4000 TU, or in the range of 0.0001 to 40 TU, or in the range of 0.001 to 40 TU per 1 mg of protein in the enzyme preparation, wherein the one tyrosine unit (TU) is equal to amount of enzyme which liberates one micromole of tyrosine equivalents from casein at 40° C. and at pH 7.

The amount of endoprotease, for example Alcalase, added to the reaction may be adjusted according to its activity so the resulting endoprotease activity in the reaction mixture may be equal to 0.01 to 1000, or 0.1 to 200, or 1 to 50 TU per 1 g of protein.

The temperature for hydrolysis may be in the range of 40° C. to 75° C., or in the range 45° C. to 70° C., or in the range of 50° C. to 65° C. Over a period of constant mixing, which may be in the range of 30 minutes to 24 hours, or in the range of 1 hour to 12 hours, or in the range of 2 hours to 8 hours, the pH of the solution decreases as the results of the hydrolysis of peptide bonds and increased number of carboxylic groups.

This allows for a high activity of exoprotease or exoprotease and endoprotease mix, for example Flavourzyme. The concentration of the exopeptidase or exoprotease and endoprotease mix may be in the range of 0.01% to 10% or in the range of 0.05% to 5%, or in the range of 0.1% to 3% expressed as a ratio of the concentration of enzyme to the concentration of protein in the reaction mixture. The concentration of the enzyme may be determined by the Bradford assay, BCA assay or other protein determination assays.

In one aspect of the invention the exopeptidase enzyme may have activity 0.001 to 2,100,000 LAPU, or 0.01 to 210,000 LAPU, or 0.1 to 21,000 LAPU (one LAPU unit is defined as amount of enzyme which liberates one micromole of p-nitroaniline from L-leucine-p-nitroanilide in one minute at 37° C. and pH 8) per 1 g of enzyme preparation of protein in enzyme solution.

In another aspect of the invention the exopeptidase enzyme may have activity 0.000005 to 1500 LAPU, or 0.00005 to 150 LAPU, or 0.0005 to 15 LAPU (one LAPU unit is defined as amount of enzyme which liberates one micromole of p-nitroaniline from L-leucine-p-nitroanilide in one minute at 37° C. and pH 8) per 1 mg of protein in the enzyme preparation.

The amount of exoprotease, for example Flavourzyme, added to the reaction may be adjusted according to its activity so the resulting exoprotease activity in the reaction mixture may be equal to 0.1 to 1500, or 1 to 500, or 5 to 100 LAPU per 1 g of protein.

The resulting mixture may then be incubated for an additional time period in the range of 1 hour to 50 hours, or in the range of 5 hours to 40 hours, or in the range of 8 hours to 30 hours at temperature in the range of 30° C. to 80° C., or in the range of 40° C. to 70° C., or in the range of 50° C. to 60° C., with constant mixing, after which the residual enzyme is thermally deactivated. With this procedure, 20% to 100%, or 25% to 70%, or 30% to 60% of the source protein may be converted into free amino acids.

The ratio of enzyme to substrate may be optimized to decrease the amount of enzyme, which is the most expensive input of the culture medium production. For example, the total amount of proteolytic enzyme used may be in the range 20% to 0.0002%, or in the range 6% to 0.05%, or in the range of 3% to 0.1% of the total amount of source protein used.

The hydrolysis by-products, including the sediment obtained from centrifugation and filtration, may be further utilized as components in subsequent applications. These by-products may be processed or refined to extract useful materials, which can serve as raw ingredients or additives in other industrial or biochemical processes, thereby enhancing the overall efficiency and sustainability of the production system. In one aspect of the invention, the sediment may be used for the production of edible products used for animal and/or human consumption.

The term “sediment” or “solid residues” refers to solid particles larger than 0.2 μm derived from the source of protein, from the enzymes or from other components entering the hydrolysis tank. In another aspect of the invention, the solid particles that may be subjected to filtration are not limited to the source of protein, enzymes or to another components entering the hydrolysis tank, but may also comprise solid particles formed in the process of preparing culture medium. It is also noted that, as an incidental effect of the solid residue removal process, particles smaller than 0.2 μm may be also removed.

In one aspect of the invention, total amino acid composition of the sediment may comprise:
alanine in a range of 0.5 wt. % to 12 wt. %, in a range of 1 wt. % to 10 wt. %, or in a range of 2 wt. % to 8 wt. % of dry mass weight;
arginine in a range of 1 wt. % to 14 wt. %, in a range of 1.5 wt. % to 12 wt. %, or in a range of 2.5 wt. % to 10 wt. % of dry mass weight;
glycine in a range of 0.5 wt. % to 10 wt. %, in a range of 1 wt. % to 8 wt. %, or in a range of 1.5 wt. % to 6 wt. % of dry mass weight;
isoleucine in a range of 0.5 wt. % to 12 wt. %, in a range of 1 wt. % to 10 wt. %, or in a range of 2 wt. % to 8 wt. % of dry mass weight;
lysine in a range of 0.5 wt. % to 11 wt. %, in a range of 1 wt. % to 9 wt. %, or in a range of 1.5 wt. % to 7 wt. % of dry mass weight;
phenylalanine in a range of 1 wt. % to 13 wt. %, in a range of 1.5 wt. % to 11 wt. %, or in a range of 2.5 wt. % to 9 wt. % of dry mass weight;
proline in a range of 0.5 wt. % to 10 wt. %, in a range of 1 wt. % to 8 wt. %, or in a range of 1.5 wt. % to 6 wt. % of dry mass weight;
serine in a range of 0.5 wt. % to 12 wt. %, in a range of 1 wt. % to 10 wt. %, or in a range of 2 wt. % to 8 wt. % of dry mass weight;
threonine in a range of 0.5 wt. % to 10 wt. %, in a range of 1 wt. % to 8 wt. %, or in a range of 1.5 wt. % to 6 wt. % of dry mass weight;
tyrosine in a range of 0.5 wt. % to 10 wt. %, in a range of 1 wt. % to 8 wt. %, or in a range of 1.5 wt. % to 6 wt. % of dry mass weight;
valine in a range of 1.5 wt. % to 14 wt. %, in a range of 2 wt. % to 12 wt. %, or in a range of 2.5 wt. % to 10 wt. % of dry mass weight;
aspartic acid in a range of 1 wt. % to 16 wt. %, in a range of 2 wt. % to 14 wt. %, or in a range of 3 wt. % to 12 wt. % of dry mass weight;
glutamic acid in a range of 1 wt. % to 18 wt. %, in a range of 3 wt. % to 16 wt. %, or in a range of 3.5 wt. % to 14 wt. % of dry mass weight;
leucine in a range of 1 wt. % to 18 wt. %, in a range of 3 wt. % to 16 wt. %, or in a range of 3.5 wt. % to 14 wt. % of dry mass weight;
histidine in a range of 0.15 wt. % to 8 wt. %, in a range of 0.5 wt. % to 6 wt. %, or in a range of 1 wt. % to 4 wt. % of dry mass weight;

In one aspect of the invention, the concentration of saccharides in the sediment may be in a range of 15 wt. % to 80 wt. %, in a range of 18 wt. % to 75 wt. %, or in a range of 20 wt. % to 70 wt. % of dry mass weight.

The method of enzymatic hydrolysis may use proteolytic enzymes in order to achieve protein hydrolysis at much milder conditions than acidic or basic hydrolysis, therefore preserving the amino acids of the original protein.

The term “proteolytic enzymes” refers to enzymes from the group of proteases, peptidases, esterases and/or any other enzyme that is capable of cleaving of peptide bonds between amino acids and/or is capable of addition of water molecule to an ester to produce alcohol or an acid.

Proteolytic enzymes may be derived from plants, animals, microorganisms, or any other appropriate source separately or in the combination thereof.

Exemplary enzymes that may be used to catalyze the breakdown of peptide bonds are Alcalase (protease from Bacillus licheniformis), Subtilisin Carlsberg (protease from Bacillus licheniformis), Flavourzyme (protease from Aspergillus oryzae), Protamex, Novo-Pro D, Protana Prime, Thermoase PC1OFNA, Protease AN Amano 100SD, Protease A Amano 2SD, Protease M Amano S D, Protease P Amano 6SD, ProteAX, Peptidase R, Alkaline Protease, Corolase 7089, Corolase 2TSN, Corolase 8000, Corolase 7000, Maxipro TNP, Maxipro FPC, Maxipro BAP, Maxipro NPU, Papain, Bromelain, Sumizyme BNP-L, Sumizyme AP-L, Sumizyme LP-G, Sumizyme FP-G, Sumizyme FL-G, Prolyve Exo L, Prolyve ALK3000L, Prolyve BS2 Liquide, Prolyve NP Liquide, Prolyve SMIX, Promod 24L, Promod 295L, Promod 327L, Promod 987MDP, Flavorpro 766MDP, Flavorpro 750MDP, Flavorpro 091MDP, ENZECO BROMELAIN 240 MCU, ENZECO FUNGAL ACID PROTEASE CONCENTRATE, ENZECO PROTEASE 400 WF, ENZECO FICIN 50K, PANOL 300 MCU or any other appropriate proteolytic enzyme, or the combination thereof.

Proteolytic enzymes may be used either alone or in combination thereof. The number of proteolytic enzymes or enzyme mixes may be in a range of 1 to 6, or in a range of 2 to 5, or in a range of 2 to 4.

In one aspect of the invention, at least one enzyme and/or enzyme mix may be used for the catalysis of the breakdown of peptide bonds in the source of protein.

The duration of the treatment by proteolytic enzymes may differ according to the source of protein used. Proteolytic enzymes may be used for a portion of time in a range of 4 hours to 40 hours, in a range of 6 hours to 35 hours, in a range of 7 hours to 30 hours, or in a range of 10 hours to 25 hours. In one aspect of the invention, the proteolytic enzymes may be used for a portion of time of at least 2 hours, at least 2.5 hours, at least 3 hours, at least 3.5 hours, at least 4 hours, at least 4.5 hours, at least 5 hours or at least 10 hours.

The temperature during the proteolysis treatment may depend on the temperature optimum of the selected proteolytic enzyme. This enzyme and/or enzymes may be used in a range of 25° C. to 80° C., in a range of 30° C. to 70° C., or in a range of 40° C. to 65° C.

Proteolytic enzymes may contain additives used to maintain pH and stability. Examples of additives include, but are not limited to water, citric acid, glycerol, potassium sorbate, sodium chloride, sorbitol, dextrin, sucrose or any other appropriate agent used for maintaining the stability and pH.

The enzymes may further comprise additives capable of maintaining stability and pH, wherein the total composition comprises additives in a range of at least 10 wt. % to 95 wt. % of total composition, 20 wt. % to 92 wt. % of total composition, 25 wt. % to 90 wt. % of total composition, or 30 wt. % to 88 wt. % of total composition.

In one aspect of the invention, the concentration of the source of protein in the reaction mixture for hydrolysis may be in the range of 1 g/l to 150 g/l, or in the range of 30 g/l to 100 g/l, or in the range of 40 g/l to 80 g/l of the reaction mixture.

In another aspect of the invention, the concentration of the source of protein in the reaction mixture for hydrolysis may be in the range of 0.5 g/l to 250 g/l, 1 g/l to 150 g/l, or in the range of 30 g/l to 100 g/l, or in the range of 40 g/l to 80 g/l of the reaction mixture.

The concentration of the enzyme may be in the range of 0.01% to 10% or in the range of 0.05% to 5%, or in the range of 0.1% to 1% expressed as a ratio of the concentration of enzyme to the concentration of protein in the reaction mixture. The concentration of the enzyme may be determined by the Bradford assay, BCA assay or other protein determination assays.

One of the approaches to characterize protein hydrolysates may be size-exclusion chromatography (SEC) combined with mass spectrometry (MS) or photo diode array (PDA) which allows us to separate the peptides from hydrolysate based on their molecular weights. The results may vary according to mobile phase composition, column type and its specification, flow rate, temperature, sample injection parameters, detector settings and/or any other parameter used.

In one aspect of the invention, proteolytic enzymes within a source of protein may yield peptides in purified protein hydrolysate with a molecular weights higher than 17 kDa, with a relative fraction size in a range 0% to 10% of the purified protein hydrolysate, in a range of 0% to 8% of the purified protein hydrolysate, or in a range of 0% to 6% of the purified protein hydrolysate.

In one aspect of the invention, proteolytic enzymes within a source of protein may yield peptides in purified protein hydrolysate with a molecular weights ranging from 6.7 to 17 kDa, with a relative fraction size in a range 2% to 35% of the purified protein hydrolysate, in a range of 4% to 30% of the purified protein hydrolysate, or in a range of 5% to 25% of the purified protein hydrolysate.

In another aspect of the invention, proteolytic enzymes within a source of protein may yield peptides in purified protein hydrolysate with a molecular weights ranging from 6.7 to 17 kDa, with a relative fraction size in a range 1% to 35% of the purified protein hydrolysate, in a range of 4% to 30% of the purified protein hydrolysate, or in a range of 5% to 25% of the purified protein hydrolysate.

In one aspect of the invention, proteolytic enzymes within a source of protein may yield peptides in purified protein hydrolysate with a molecular weights ranging from 1.7 kDa to 6.7 kDa, with a relative fraction size in a range 5% to 40% of the purified protein hydrolysate, in a range of 10% to 35% of the purified protein hydrolysate, or in a range of 15% to 30% of the purified protein hydrolysate.

In another aspect of the invention, proteolytic enzymes within a source of protein may yield peptides in purified protein hydrolysate with a molecular weights ranging from 1.7 kDa to 6.7 kDa, with a relative fraction size in a range 1% to 40% of the purified protein hydrolysate, in a range of 10% to 35% of the purified protein hydrolysate, or in a range of 15% to 30% of the purified protein hydrolysate.

In one aspect of the invention, proteolytic enzymes within a source of protein may yield amino peptides in purified protein hydrolysate with a molecular weights ranging from 1 kDa to 1.7 kDa, with a relative fraction size in a range 5% to 40% of the purified protein hydrolysate, in a range of 10% to 35% of the purified protein hydrolysate, or in a range of 20% to 30% of the purified protein hydrolysate.

In another aspect of the invention, proteolytic enzymes within a source of protein may yield amino peptides in purified protein hydrolysate with a molecular weights ranging from 1 kDa to 1.7 kDa, with a relative fraction size in a range 1% to 40% of the purified protein hydrolysate, in a range of 10% to 35% of the purified protein hydrolysate, or in a range of 20% to 30% of the purified protein hydrolysate.

In one aspect of the invention, proteolytic enzymes within a source of protein may yield free amino acids and/or peptides in purified protein hydrolysate with a molecular weights less than 1 kDa, with a relative fraction size in a range 20% to 70% of the purified protein hydrolysate, in a range of 22% to 60% of the purified protein hydrolysate, or in a range of 25% to 55% of the purified protein hydrolysate.

In another aspect of the invention, proteolytic enzymes within a source of protein may yield free amino acids and/or peptides in purified protein hydrolysate with a molecular weights less than 1 kDa, with a relative fraction size in a range 20% to 100% of the purified protein hydrolysate, in a range of 22% to 70% of the purified protein hydrolysate, or in a range of 25% to 60% of the purified protein hydrolysate.

In one aspect of the invention, proteolytic enzymes within a source of protein may yield free amino acids in purified protein hydrolysate with a relative fraction size in a range of 30% to 50% of the purified protein hydrolysate, in a range of 35% to 45% of the purified protein hydrolysate, or in a range of 38% to 42% of the purified protein hydrolysate.

In another aspect of the invention, proteolytic enzymes within a source of protein may yield free amino acids in purified protein hydrolysate with a relative fraction size in a range of 20% to 100% of the purified protein hydrolysate, in a range of 25% to 70% of the purified protein hydrolysate, or in a range of 30% to 60% of the purified protein hydrolysate.

In one aspect of the invention, an additional method may be used to further separate the small peptides with molecular weights ranging from 1000 Da to 6500 Da, or 600 Da to 1000 Da, or 300 Da to 600 Da, or molecular weights lower than 300 Da.

In one aspect of the invention, proteolytic enzymes within a source of protein may yield amino acids and/or peptides in purified protein hydrolysate with a molecular weights ranging from 1000 Da to 6500 Da, with a relative fraction size in a range of 5% to 50%, in a range of 10% to 40%, or in a range of 15% to 35% of the measured fractions.

In one aspect of the invention, proteolytic enzymes within a source of protein may yield amino acids and/or peptides in purified protein hydrolysate with a molecular weights ranging from 1000 Da to 6500 Da, with a relative fraction size in a range of 1% to 70%, in a range of 5% to 40%, or in a range of 10% to 35% of the measured fractions.

In one aspect of the invention, proteolytic enzymes within a source of protein may yield amino acids and/or peptides in purified protein hydrolysate with a molecular weights ranging from 600 Da to 1000 Da, with a relative fraction size in a range of 10% to 70%, in a range of 15% to 60%, or in a range of 20% to 50% of the measured fractions.

In one aspect of the invention, proteolytic enzymes within a source of protein may yield amino acids and/or peptides in purified protein hydrolysate with a molecular weights ranging from 600 Da to 1000 Da, with a relative fraction size in a range of 1% to 60%, in a range of 2% to 40%, or in a range of 5% to 30% of the measured fractions.

In one aspect of the invention, proteolytic enzymes within a source of protein may yield amino acids and/or peptides in purified protein hydrolysate with a molecular weights ranging from 300 Da to 600 Da, with a relative fraction size in a range of 10% to 65%, in a range of 15% to 55%, or in a range of 20% to 45% of the measured fractions.

In one aspect of the invention, proteolytic enzymes within a source of protein may yield amino acids and/or peptides in purified protein hydrolysate with a molecular weights ranging from 300 Da to 600 Da, with a relative fraction size in a range of 1% to 60%, in a range of 2% to 40%, or in a range of 5% to 30% of the measured fractions.

In one aspect of the invention, proteolytic enzymes within a source of protein may yield amino acids and/or peptides in purified protein hydrolysate with a molecular weights less than 300 Da, with a relative fraction size in a range of 10% to 90%, in a range of 15% to 80%, or in a range of 20% to 70% of the measured fractions.

In one aspect of the invention, proteolytic enzymes within a source of protein may yield amino acids and/or peptides in purified protein hydrolysate with a molecular weights less than 300 Da, with a relative fraction size in a range of 20% to 100%, in a range of 30% to 80%, or in a range of 40% to 70% of the measured fractions.

In another aspect of the invention, a complementary method may be used to further separate the small peptides with molecular weights ranging from 1500 Da to 7000 Da, 1000 Da to 1500 Da, 300 Da to 1000 Da, or molecular weights lower than 300 Da to provide detailed peptide molecular weight distribution evaluation.

In one aspect of the invention, proteolytic enzymes within a source of protein may yield peptides in purified protein hydrolysate with a molecular weights ranging from 1500 Da to 7000 Da, with a relative fraction size in a range of 1% to 50%, in a range of 5% to 40%, or in a range of 10% to 30% of the measured fractions.

In one aspect of the invention, proteolytic enzymes within a source of protein may yield peptides in purified protein hydrolysate with a molecular weights ranging from 1000 Da to 1500 Da, with a relative fraction size in a range of 1% to 60%, in a range of 2% to 50%, or in a range of 5% to 40% of the measured fractions.

In one aspect of the invention, proteolytic enzymes within a source of protein may yield peptides in purified protein hydrolysate with a molecular weights ranging from 300 Da to 1000 Da, with a relative fraction size in a range of 1% to 60%, in a range of 2% to 40%, or in a range of 5% to 30% of the measured fractions.

In one aspect of the invention, proteolytic enzymes within a source of protein may yield amino acids and/or peptides in purified protein hydrolysate with a molecular weights less than 300 Da, with a relative fraction size in a range of 20% to 100%, in a range of 30% to 80%, or in a range of 40% to 70% of the measured fractions.

In one aspect of the invention, the water source may comprise distilled water, demineralized water, deionized water and/or tap water. In one aspect of the invention, the water source may be characterized by conductivity in a range of 1 μS·cm⁻¹ to 600 μS·cm⁻¹, in a range of 5 μS·cm⁻¹ to 100 μS·cm⁻¹ or in a range of 10 μS·cm⁻¹ to 50 pS·cm⁻¹.

In one aspect of the invention, the proteolytic enzymes may be immobilized on a solid support. This aspect sterically prevents the molecules of the enzyme from breaking each other down and allows the enzyme to be separated from the reaction mixture after the reaction and used again. The solid support may be present in the form of solid carriers suspended in the reaction mixture, or a solid structure with a large surface area, such as a sponge or fibrous structure, through which the reaction mixture is perfused. The enzyme may also be added in soluble (free) form. After hydrolysis is complete, the resulting protein hydrolysate is separated from the solid support with immobilized enzyme by simply draining the reaction vessel (in the case of large solid structure) or removing the enzyme on solid support by filtration or sedimentation (in the case of suspended carriers). The reaction vessel may be formed, for example, by a hydrolysis tank. The filtration step may also remove any solid residues from the source protein, such as cell wall debris. Free enzymes may be removed from the protein hydrolysate by ultrafiltration or deactivated with elevated temperature when hydrolysis is complete. Ultrafiltration of the protein hydrolysate may additionally remove any larger peptide chains which were not digested by the enzyme; these relatively larger peptide chains may not be metabolized by the cells and cause harm to them and therefore their removal may be beneficial. The temperature elevation used to deactivate the enzyme may also sterilize the resulting protein hydrolysate.

The protein hydrolysate may be thermally treated at the end of hydrolysis to deactivate enzymes and kill microorganisms. In one aspect of the invention, this treatment may take place at a lower temperature settings in the range of 80° C. to 120° C., in the range of 85° C. to 100° C. or in the range of 90° C. to 95° C. for time in the range of 15 minutes to 180 minutes or in the range of 20 minutes to 120 minutes or in the range of 25 minutes to 60 minutes. In another aspect of the invention, the thermal treatment may be performed at a high temperature in the range 100° C. to 180° C., in the range of 120° C. to 170° C., or in the range of 130° C. to 160° C., or in the range of 135° C. to 150° C. for a time period in the range of 1 second to 60 seconds, in the range of 3 seconds to 30 seconds, or in the range of 5 seconds to 20 seconds, or in the range of 10 seconds to 15 seconds. The low temperature method may be performed in a hydrolysis tank, wherein the both methods may be performed in the hydrolysis tank configured as flash pasteurizer or another suitable continuous flow heating device.

If the enzyme is removed by ultrafiltration, it may retain at least partial catalytic activity and thus may be recycled for another round of hydrolysis. Ultrafiltration or thermal deactivation may also be used to remove active enzyme molecules from hydrolysates prepared by immobilized enzymes, in the event that some of the enzyme detaches from the solid support and dissolves into the reaction mixture.

The solid support may be formed by, for example, silica, epoxide resin, cellulose, chitosan, glass wool, alginate, or by other appropriate materials. The solid support may be in the form of porous or solid beads, sponge, fibers, or another suitable configuration. The solid support may have a large surface area to volume ratio to allow the binding of a large amount of enzyme. For example, beads of porous silica or any other suitable material with a diameter in the range of 1 μm to 10,000 μm, or in the range of 10 μm to 1,000 μm, or in the range of 20 μm to 500 μm, may be used as a solid support for enzyme immobilization. Immobilization may be achieved, for example, by functionalizing the silica bead surface with amino groups and using a crosslinking agent, such as glutaraldehyde, to bind the enzyme to the solid support. Other functional groups, like aldehyde or epoxy groups, may also be used for enzyme immobilization. The amino groups in this aspect of the invention are covalently bonded to glutaraldehyde, after which excess glutaraldehyde is removed and the enzyme is added. The amino groups on the surface of the enzyme then bind the remaining free aldehyde groups of the glutaraldehyde molecules on the silica bead surface. The immobilization may be performed in water or a suitable aqueous buffer. Due to the porous nature and large surface area of the silica beads, a relatively high amount of enzyme may be immobilized relative to the weight of the solid support.

Water may be used to dissolve the source of protein for hydrolysis. Some proteins may require a buffer to adjust the pH to a level where they have better solubility. The pH may be in the range of 2 to 12, or in the range of 5 to 10, or in the range of 6 to 8.5. A very dilute buffer, or no buffer at all, may be used so that the resulting protein hydrolysate may be added to the final culture media at high concentrations while minimizing its impact on media osmolality.

The buffer may include, for example, phosphate buffer, bicarbonate buffer, tris HCl buffer, borate buffer, glycine-NaOH buffer, Good's buffer or any other appropriate buffer, or a combination thereof.

In one aspect of the invention, a concentration of potassium phosphate buffer in a range of 1 mM to 100 mM, in a range of 10 mM to 40 mM or in a range of 15 mM to 35 mM may be used for pH adjustment to dissolve soy protein to a concentration in a range of 1 g/l to 150 g/l, in a range of 20 g/l to 100 g/l, in a range of 30 g/l to 80 g/l, in a range of 40 g/l to 70 g/l, in a range of 50 g/l to 65 g/l or in a range of 55 g/l to 60 g/l. In another aspect of the invention, the soy protein is dissolved in distilled water to a concentration in a range of 1 g/l to 150 g/l, in a range of 20 g/l to 100 g/l, in a range of 30 g/l to 80 g/l, in a range of 30 g/l to 80 g/l, in a range of 40 g/l to 70 g/l, in a range of 50 g/l to 65 g/l or in a range of 55 g/l to 60 g/l, where the concentration is defined as the amount of source of protein per liter of the reaction multiplied by the percentage protein content in the source of protein.

In another aspect of the invention, a concentration of potassium phosphate buffer in a range of 1 mM to 100 mM, in a range of 10 mM to 40 mM or in a range of 15 mM to 35 mM may be used for pH adjustment to dissolve soy protein to a concentration in a range of 0.5 g/l to 250 g/l, or in a range of 1 g/l to 150 g/l, or in a range of 30 g/l to 100 g/l, or in a range of 40 g/l to 80 g/l. In another aspect of the invention, the soy protein is dissolved in distilled water to a concentration in a range of 0.5 g/l to 250 g/l, or in a range of 1 g/l to 150 g/l, or in a range of 30 g/l to 100 g/l, or in a range of 40 g/l to 80 g/l, where the concentration is defined as the amount of source of protein per liter of the reaction multiplied by the percentage protein content in the source of protein.

Other concentrations of the source of protein may be used, however, very high concentrations of this source lead to incomplete dissolving of the protein and formation of a highly viscous colloidal solution, presenting problems for the hydrolysis and further processing, while low concentrations of protein may limit the speed of the hydrolysis reaction. To ensure the best dissolution of the proteins in the reaction mixture a heat-treatment may be used. Below boiling temperatures may be used for extended periods of time in order to significantly increase the content of dissolved proteins and to deactivate potential inhibitors of proteases and other antinutritional compounds.

In one aspect of the invention, the source of protein may be added at a higher concentration than the maximum soluble concentration. This additional protein may be dissolved after the protein concentration in the reaction mixture is decreased due to its hydrolysis by the enzyme. This results in high concentration of available substrate during the entire process, potentially improving hydrolysis efficiency. Multiple cycles of substrate addition into the same reaction mixture may be performed. In one aspect of the invention a base or a suitable buffer may be added to counteract changes in pH and keep the enzyme in its pH optimum or a pH stat may be used.

A parameter by which the conversion of source of protein into bioavailable products for animal cells may be evaluated, is the degree of hydrolysis (DH), defined as the percentage of peptide bonds in the source of protein that are hydrolyzed during the reaction. DH can be determined as the difference of amino nitrogen (AN) of hydrolysed substrate and amino nitrogen of substrate before hydrolysis (ANO) multiplied by factor (F) and divided by total nitrogen (TN). AN may be determined by formol titration of the hydrolysate sample, ANO may be determined by formol titration of the substrate solution before the process of hydrolysis, TN may be determined by Kjeldahl method. Factor F is a value calculated from empirical data based on amino acids composition of the particular source of protein and it represents the ratio of total nitrogen to alpha amino nitrogen in the sample. A higher degree of hydrolysis corresponds to a larger percentage of the source protein converted into free amino acids or short peptides, which are usable by mammalian cells as nutrition. Mammalian cells are generally incapable of absorbing and digesting proteins and longer peptides. Peptides longer than four amino acids, or in other words heavier than approximately 500 Daltons, have poor absorption by mammalian cells. In various aspects of the invention, the amount of the source of protein in the range of 20% to 100%, in the range of 30% to 75%, in the range of 35% to 70% or in the range of 40% to 65% may be converted into free amino acids, expressed as mass concentration of amino acids to mass concentration of protein. The degree of hydrolysis, meaning the percentage of peptide bonds that undergo hydrolysis out of the total amount of peptide bonds present in the substrate at the start of the reaction, may be in the range of 10% to 70%, in the range 20% to 60% or in the range of 25% to 50%.

Proteolytic enzymes are classified according to the basis of amino acid group present on the active site as serine proteases (subtilisin, trypsin), cysteine proteases (papain-like, trypsin-like), aspartic proteases (pepsin, cathepsin D), glutamic proteases (eqolisin), threonine proteases (ornithine, acetyltransferase) and metalloproteases (Myxobacter I and II). Another classification divides enzymes according to their enzymatic function into exoproteases and endoproteases. Exoproteases cleave the protein or peptide chains at the N or C terminal ends, whereas endoproteases can cleave peptide bonds in the middle of the protein or peptide chain. Exoproteases are classified according to the mechanism of action on aminopeptidases that act on the N terminal end and carboxypeptidases that act on the C terminal end. The present examples of proteases acting on the active side of related amino acids are not limited to the listed exemplary proteases.

In one aspect of the invention, a combination of endoproteases and exoproteases may be used, since endoproteases may create more free ends of peptide chains, increasing the efficiency of exoproteases, and exoproteases are more efficient in hydrolyzing the protein to single amino acids.

In one aspect of the invention, endoproteases and exoproteases may be used sequentially in this order to maximize hydrolysis efficiency.

In one aspect of the invention, the terms “endoprotease” and “endopeptidase” are used interchangeably and may refer to enzymes that cleave peptide bonds within a polypeptide chain. Similarly, the terms “exoprotease” and “exopeptidase” are used interchangeably and may refer to enzymes that cleave peptide bonds from the terminal ends of a polypeptide chain.

In one aspect of the invention, additional enzymes may be added to the reaction mixture after the beginning of hydrolysis. This may be done with another enzyme or the same enzyme, mainly in order to counteract the gradual decrease in its enzymatic activity due to degradation of the enzyme molecule. In one aspect of the invention, enzymes with a higher pH optimum may be added at the start of the hydrolysis, when pH is higher, and enzymes with a lower pH optimum may be added later, when the pH is lower, thus maximizing the efficiency of the respective enzymes. The pH tends to decrease naturally during hydrolysis due to the increase in the number of carboxylic groups.

In another aspect of the invention, additional sources of protein may be added to the reaction mixture after the beginning of hydrolysis. The advantages of this aspect may be, for example, easier dispersion and dissolution of additional sources of protein when the previous amount of source of protein is at least partially hydrolyzed.

The addition of enzyme or substrate after the beginning of the hydrolysis process may be performed in a fed-batch (wherein additional reagents are added to the reaction mixture, and subsequently the whole reaction batch is harvested) or semi-continuous (wherein a portion of the reaction mixture, or certain additional reagents within the reaction mixture, is periodically removed and replaced with fresh components) or in continuous (wherein addition to and harvesting from the reaction mixture are both done continuously) reaction mode.

Regardless of whether immobilized or free enzyme is used, sufficient mixing of the reaction mixture is important to achieve high efficiency. In the case of immobilized enzymes, this applies to both the enzyme immobilization and protein hydrolysis steps. In one aspect of the invention, in the case of immobilized enzymes, mixing methods that minimize mechanical damage to the solid carriers should be used. These may include roller mixing, shaking, or low-shear impellers such as hydrofoil or elephant ear impellers. In the case of enzymes immobilized to a large solid support, sufficient perfusion of the support with the reaction mixture must be assured.

The mixing of the source of protein, e.g. protein isolate, with water, dissolving the source of protein and the process of hydrolysis itself may be performed in the hydrolysis tank at a laboratory or industrial scale.

In one aspect of the invention, the hydrolysis tank may be configured to provide an environment for the hydrolysis reaction. The hydrolysis tank may comprise a main body constructed from at least one material selected from stainless steel, glass-lined steel, titanium, polyethylene, polypropylene, polytetrafluoroethylene or any other suitable materials. The main body may comprise various shapes, such as cylindrical or rectangular or any other suitable geometries. The hydrolysis tank may comprise insulation configured as an outer jacket of the hydrolysis tank, wherein the space between the outer jacket and the wall of the hydrolysis tank may be filled with an appropriate insulation material or medium. The hydrolysis tank may further comprise at least one input and at least one output for loading and unloading the ingredients. The input of the hydrolysis tank may be configured as a shaft or funnel, wherein the shaft or funnel may be used for loading the ingredients. The hydrolysis tank may further comprise a heating system configured to heat the inner environment of the hydrolysis tank. The hydrolysis tank may comprise mixing mechanisms comprising at least one stirrer, paddle or any other instrument capable of mixing the protein hydrolysate. The sealing mechanisms of the hydrolysis tank may comprise materials such as silicone, ethylene propylene diene monomer, and polytetrafluoroethylene. The hydrolysis tank may be configured to withstand a maximum temperature of at least 80° C., at least 90° C., at least 100° C., at least 105° C., at least 110° C., at least 120° C., or at least 150° C. The hydrolysis tank may further comprise auxiliary components selected from the group of pumps, pressure sensors, flow meters, valves and means for monitoring the hydrolysis reaction.

The volume of the hydrolysis tank may be in the range of 0.1 1 to 100,000 1, or in the range of 0.3 1 to 15,000 1, or in the range of 1 1 to 5,000 1, or in the range of 0.3 1 to 20,000 1, or in the range of 1 1 to 10,000 1.

In one aspect of the invention, the hydrolysis tank may be equipped with different types of sensors, for example, thermal sensor, pH probe, conductometer, or any other type of appropriate sensor according to the needs of the process of hydrolysis. The pH may be measured by various methods and devices comprising potentiometry, colorimetry, spectrophotometry, ion-selective electrodes, conductometry or any other measuring technique and/or device. The temperature in the reaction vessel may be measured by various devices comprising resistance temperature detector, thermocouple, digital thermometer with insertion probe, infrared thermometer with fiber optic probe or any other appropriate device.

A sampling system may be used for precise monitoring of the degree of hydrolysis a sampling system may be used. The degree of hydrolysis may be monitored by titration and/or by absorbance measurement, for example at a wavelength in a range of 190 nm to 350 nm, or in a range 190 nm to 230 nm.

Water may enter the hydrolysis tank through a water purification unit. In one aspect of the invention, a water purification unit may provide at least one selected purification process from the group of reverse osmosis, deionization, electrodeionization, electrodialysis and distillation.

The mixing may be provided by the appropriate stirring unit that may comprise, for example, a paddle impeller. In some aspects, an elephant-ear impeller may be used. The outer diameter of the stirrer or impeller may be in the range of 1/10 to 9/10 of the inner reactor diameter, or in the range of 3/10 to 8/10 of the inner reactor diameter, or in the range of 4/10 to 7/10 of the inner reactor diameter. As an example the outer diameter of stirrer/impeller may be ⅔ of the inner reactor diameter. The stirrer or impeller may be located in the center of the hydrolysis tank or outside of the center of the hydrolysis tank.

In another aspect of the invention, the mixing in the hydrolysis tank may be provided by the appropriate stirring unit that may comprise, for example, a paddle impeller. In some aspects, an elephant-ear or rushton-like impeller may be used. The outer diameter of the stirrer or impeller may be in the range of 1/10 to 9/10 of the inner reactor diameter, or in the range of 3/10 to 8/10 of the inner reactor diameter, or in the range of 4/10 to 7/10 of the inner reactor diameter. As an example the outer diameter of stirrer/impeller may be ⅔ of the inner reactor diameter. The stirrer or impeller may be located in the center of the hydrolysis tank or outside of the center of the hydrolysis tank. The hydrolysis tank may be equipped with baffles or other suitable features to prevent vortex formation and improve mixing efficiency.

The reaction components may be added to the hydrolysis tank manually, or automatically by using a conveyor, loading tank or any other appropriate device used for transfer of reaction components. The source of protein may be in a liquid solution or in the form of a powder.

The automation system may use various types of pumps for feeding enzymes at precise temperatures during hydrolysis. Each enzyme may be pumped at a certain temperature. Both parameters may be set ahead of the operation in an automation profile setting for the hydrolysis process.

The term “reaction components” may comprise a source of protein, proteolytic enzymes, or any other appropriate component necessary for effective hydrolysis by proteolytic enzymes.

The hydrolysis tank may be equipped with a temperature control unit (TCU) that is configured to maintain the temperature of the culture medium at the desired temperature range. The desired temperature setpoint may be changed at least once during the process. The TCU may be equipped with heating and/or cooling exchangers and at least one pump.

In one aspect of the invention, the temperature may be controlled automatically through a cascade control system, wherein the tank temperature acts as the primary control variable, and the temperature control unit (TCU) responds to maintain the hydrolysis tank at the desired setpoint. The cascade control system continuously adjusts the TCU heating and/or cooling output based on real-time feedback from the hydrolysis tank to ensure stable and precise temperature regulation during hydrolysis that takes place in the hydrolysis tank.

In one aspect of the invention, the loading tank may be configured to provide proteolytic enzymes in liquid form into the hydrolysis tank. The loading tank may be configured to maintain the optimal conditions necessary for preserving the stability and activity of stored enzymes. The loading tank may comprise a main body constructed from at least one material selected from high-grade stainless steel, glass-lined steel, titanium, polyethylene, polypropylene, polytetrafluoroethylene or any other suitable materials. The main body may comprise various shapes, such as cylindrical or rectangular or any other suitable geometries. The interior surface of the loading tank may be polished to a mirror finish to minimize adhesion of enzyme residues and facilitate easy cleaning. The loading tank may be equipped with a cooling system to maintain a consistent temperature, essential for enzyme stability. The loading tank may comprise multiple temperature sensors to continuously monitor the internal temperature. The loading tank may be coupled with the hydrolysis tank, wherein the hydrolysis tank may further comprise at least one input and at least one output for loading and unloading the enzymes. The input of the loading tank may be configured as a shaft and/or funnel, wherein the shaft and/or funnel may be used for loading of the proteolytic enzymes. The sealing mechanisms of the loading tank may comprise materials such as silicone, ethylene propylene diene monomer, and polytetrafluoroethylene and/or any other appropriate material.

The loading tank 126 may be made, for example, of stainless steel or glass. The volume of the loading tank may be in the range of 100 ml to 5 m³, or in the range of 2 1 to 3 m³, or in the range of 500 1 to 1 m³.

In one aspect of the invention, another loading tank 126 may be configured to provide a source of protein into the hydrolysis tank with the same composition as the loading tank for the proteolytic enzymes.

Many plant sources of protein comprise inositol hexaphosphate, which is an important compound found in plants and is often a crucial part of a plant metabolism. Its salt form, phytin, is the main storage compound of phosphate in plants. The inositol hexaphosphate or its derivatives and/or any related form of those compounds comprising phosphate from plant sources may significantly influence the downstream processes of the invention as well as metazoan cell proliferation and viability of the cell. In order to regulate the content and/or concentration of such compounds, the 4 aspects are disclosed.

The first aspect disclosed herein relates to the use of enzymes having phytase activity derived from an animal, plant, or microorganism source or any other appropriate source. Enzymes having phytase activity may be used separately or in the combination thereof.

The term “enzyme having phytase activity” refers to the enzymes from the group of phytases, phosphatases and/or any other enzymes capable of cleaving phosphate ester bonds.

Activity of enzymes having phytase activity may differ according to the source of protein. Enzymes having phytase activity may have different activity at different pH and/or temperature conditions, which may be measured by various methods and devices including potentiometry, colorimetry, spectrophotometry, ion-selective electrodes, conductometry and/or any other measuring technique and/or device. Temperature of the reaction mixture may be monitored in real time by various devices including a resistance temperature detector, thermocouple, digital thermometer with insertion probe, infrared thermometer with fiber optic probe or any other appropriate device.

The enzyme kinetics of the reaction may be calculated based on the Michaelis-Menten model, which provides a foundational equation for describing the rate of enzymatic reaction to provide a product from a substrate. This model is integral for understanding of the relationship between substrate concentration and reaction velocity, and its incorporation allows for precise determination of key kinetic parameters, such as the maximum reaction velocity (Vₘₐₓ) and the Michaelis constant (Kₘ).

Enzymes having phytase activity may contain additives used to maintain pH and stability. Examples of additives include, but are not limited to water, citric acid, glycerol, potassium sorbate, sodium chloride, sorbitol, dextrin, sucrose or any other appropriate agent used for maintaining the stability and pH.

Enzymes having phytase activity may be used either individually or in combination, wherein the combination may comprise use of at least 2 or more enzymes having phytase activity.

The duration of the treatment by enzymes having a phytase activity may differ according to the source of protein used. The duration of the treatment by enzymes having phytase activity may be finished at different times, which may be in a range of 5 minutes to 320 minutes, in a range of 10 minutes to 180 minutes, or in a range of 30 minutes to 120 minutes.

The temperature of the treatment by enzymes having a phytase activity may be in a range of 20° C. to 70° C., in a range of 25° C. to 65° C., or in a range of 30° C. to 60° C.

Enzymes having phytase activity may create complexes with a substrate. The substrate may comprise any inositol hexaphosphate or its derivatives that may undergo cleaving of phosphate ester bound connecting phosphate groups with inositol or its derivatives, in the presence of water. The function of enzymes having phytase activity according to this aspect of the invention is not limited to said substrates.

Enzymes having phytase activity may catalyze release of at least one organophosphate group from inositol hexaphosphate or its derivatives and may result in free inorganic phosphate and series of lower phosphoric esters as inositol pentaphosphate, inositol tetraphosphate, inositol triphosphate, inositol diphosphate, inositol monophosphate intermediates or inositol or their combination thereof.

After each cleavage of phosphate ester bond and release of inorganic phosphate and series of lower phosphoric esters as inositol pentaphosphate, inositol tetraphosphate, inositol triphosphate, inositol diphosphate, inositol monophosphate intermediates, inositol or their combination thereof, the enzyme having phytase activity may then bind to another inositol hexaphosphate or its derivatives or any related form of those compounds for further hydrolysis in the presence of water.

In another aspect of the invention, additional sources of protein may be added to the reaction mixture after the beginning of hydrolysis. The advantages of this aspect may be, for example, easier dispersion and dissolution of additional sources of amino acids when the previous amount of source of protein is at least partially hydrolyzed.

Addition of enzymes having phytase activity in a solid or liquid state or in a combination thereof, into hydrolysis tank containing a source of protein and water source from loading tank leads to cleavage of phosphate ester bonds and release of molecules in the specific pH and temperature.

In one aspect of the invention, the hydrolysis tank may be composed of various materials and specific volumes and may be coupled with a water purification unit, loading tank and may comprise a stirring unit.

In one aspect of the invention, the loading tank may be configured to provide a combination of enzymes having phytase activity in dry or liquid form or in combination thereof and proteolytic enzymes and/or source of protein into the hydrolysis tank.

In another aspect of the invention, the cultivation system may comprise two or more hydrolysis tanks. When using two or more hydrolysis tanks, they may be referred to, for example, as first hydrolysis tank, second hydrolysis tank and third hydrolysis tank. Notations of hydrolysis tanks may be chosen according to the selected number of hydrolysis tanks. The first hydrolysis tank may be configured for hydrolysis of the source of protein by proteolytic enzymes, and the second hydrolysis tank may be configured for the hydrolysis of inositol hexaphosphate and/or its derivatives by enzymes having phytase activity. All of the hydrolysis tanks may be coupled with a mixing tank, water purification unit, at least one loading tank, at least one pump and/or at least one filtration unit.

In one aspect of the invention, the hydrolysis tank may be configured to perform both hydrolysis of the source of protein by proteolytic enzymes and hydrolysis of inositol hexaphosphate and/or its derivatives.

In one aspect of the invention, the concentration of enzymes having phytase activity may differ according to concentration of inositol hexaphosphate contained in the source of protein. The concentration of the enzymes having phytase activity may be in the range of 0.00001% to 5% or in the range of 0.0001% to 2%, or in the range of 0.0005% to 0.5% expressed as a ratio of the concentration of enzymes having phytase activity to the concentration of inositol hexaphosphate in the reaction mixture. The concentration of the enzyme may be determined by the Bradford assay, BCA assay or other protein determination assays. The concentration of inositol hexaphosphate or its derivatives may be determined by electrophoresis, liquid chromatography, enzymatic assay or any other suitable analytical method.

The enzyme having phytase activity may have phytase activity in the range of 0.0001 to 3,000,000 FTU, or in the range of 0.001 to 300,000 FTU, or in the range of 0.01 to 30,000 FTU (one phytase unit is equal to amount of enzyme that liberates one micromole of phosphate in one minute from phytate at 37° C. and pH 5.5) per 1 gram of the enzyme preparation.

The amount of enzyme having phytase activity, for example Maxamyl, added to the reaction may be adjusted according to its activity so the resulting exoprotease activity in the reaction mixture may be equal to 0.001 to 1000, or 0.01 to 500, or 1 to 100 FTU per 1 gram of protein source.

The efficiency of cleavage by enzymes having phytase activity may be regulated by pH or temperature change. Efficiency of cleavage may be improved by addition of a pH regulating substance to obtain pH in a range of 2 to 10, in a range of 3 to 9, in a range of 4 to 8, in a range of 5 to 9, or in a range of 6 to 7, or in a range of 5.6 to 8.5, or in a range of 5.2 to 7.5, or in a range of 5.1 to 6, wherein the substance can be an inorganic molecule comprising HCl, NaOH and/or NH₄OH or any other appropriate pH regulating compound. Efficiency of cleavage may be improved by changing the temperature. The temperature may be in a range of 20° C. to 70° C., in a range of 25° C. to 65° C., or in a range of 30° C. to 60° C.

In one aspect of the invention, the use of enzymes having phytase activity on protein hydrolysate may result in a modified protein hydrolysate. As used herein, the term “modified protein hydrolysate” refers to protein hydrolysate comprising cleaved or precipitated inositol hexaphosphate and its derivatives or their combination thereof.

The modified protein hydrolysate may be subjected to thermal treatment at the end of hydrolysis to deactivate enzymes having phytase activity and/or kill microorganisms. In one aspect of the invention, this treatment may be at low temperature in the range of 80° C. to 120° C., in the range of 85° C. to 100° C. or in the range of 90° C. to 95° C. for a time in the range of 15 minutes to 180 minutes or in the range of 20 minutes to 120 minutes or in the range of 25 minutes to 60 minutes.

In another aspect of the invention, thermal treatment may be performed at a high temperature in the range 80° C. to 160° C., in the range of 100° C. to 155° C., or in the range of 110° C. to 150° C. for a time is in the range of 1 second to 600 seconds, in the range of 3 seconds to 300 seconds, or in the range of 5 seconds to 60 seconds. The thermal treatment may be performed at a low temperature in a hydrolysis tank, wherein the both thermal treatments (at low and/or high temperature) may be performed in the hydrolysis tank configured as flash pasteurizer or another suitable continuous flow heating device.

In another aspect of the invention, thermal treatment may be performed at a high temperature in the range 100° C. to 180° C., in the range of 120° C. to 170° C., or in the range of 130° C. to 160° C., or in the range of 135° C. to 150° C. for a time period in the range of 1 second to 60 seconds, in the range of 3 seconds to 30 seconds, or in the range of 5 seconds to 20 seconds, or in the range of 10 seconds to 15 seconds. The low temperature method may be performed in a hydrolysis tank, wherein the both methods may be performed in the hydrolysis tank configured as flash pasteurizer or another suitable continuous flow heating device.

If the enzyme is removed by ultrafiltration, it may retain at least partial catalytic activity and thus may be recycled for another round of hydrolysis. Ultrafiltration or thermal deactivation may also be used to remove active enzyme molecules from hydrolysates prepared by immobilized enzymes, in the event that some of the enzyme detaches from the solid support and dissolves into the reaction mixture.

In one aspect of the invention, the use of proteolytic enzymes and enzymes having phytase activity may be in one of two orders. In the first order, enzymes having phytase activity may be added to the hydrolyzed source of protein and water in the hydrolysis tank. The efficiency of cleavage of phosphate ester bonds may be regulated by pH and temperature changes. In the second order, proteolytic enzymes may be added to the source of protein, water and to previously cleaved phosphate and series of lower phosphoric esters as inositol pentaphosphate, inositol tetraphosphate, inositol triphosphate, inositol diphosphate, inositol monophosphate, inositol, intermediates of inositol derivatives or their combination thereof in the hydrolysis tank to generate protein hydrolysate free of inositol hexaphosphate and its derivatives. The second order is less preferable due to the disadvantageous step of pH change especially if the enzyme used has a pH optimum in the acidic region.

In one aspect of the invention, solid residues may be removed from modified protein hydrolysate by a filtration unit.

In one aspect of the invention, the solid residues may be further used for the production of edible product, wherein the edible product may be used for human or animal consumption.

In one aspect of the invention, the hydrolysis tank may be connected to the filtration unit by pump. This pump may be used for the transfer of protein hydrolysate to the filtration unit.

In one aspect of the invention, the filtration unit may comprise at least one filter selected from the group of membrane filters, depth filters, mesh filters, activated carbon filters, ceramic filters, ultrafiltration filters, nanofiltration filters, ion exchange filters, crossflow (tangential flow) filters, adsorption filters or fiber filters. The filter of the filtration unit may comprise at least one material selected from the group of cellulose, glass fiber, polyethersulfone (PES), polyvinylidene fluoride (PVDF), nylon, polypropylene, polyethylene terephthalate (PET), polytetrafluoroethylene (PTFE), polyacrylonitrile (PAN), polyvinyl chloride (PVC), stainless steel, silica, alumina, silicon carbide, titanium dioxide, titanium carbide, silicon carbide, zeolites, or synthetic polymers. The filter may be housed in a housing configured to cover the whole filter, wherein the housing may comprise at least one material selected from the group of stainless steel, polycarbonate, polyethylene, or other suitable biocompatible and sterilizable materials. If the filtration unit is composed of multiple filters or includes filters with non-uniform pore size distribution across the depth of the filter, the filters may be arranged so that the filters with largest pores and/or, in the case of filters with non-uniform pore size distribution, the side with the largest pores, may be located upstream (in the direction of the hydrolysis tank) and the filters with the smallest pores and/or the side with the smallest pores may be located downstream (in the direction of the mixing tank within the filtration unit. In case of using multiple filters or using filters with non-uniform pore size distribution across the depth of the filter, the maximal pore size of the last filter used of the filtration unit may be in a range of in a range of 0.1 μm to 1 μm, in a range of 0.2 μm to 1 μm, in a range of 0.3 μm to 1 μm, in a range of 0.4 μm to 1 μm, in a range of 0.5 μm to 1 μm, in a range of 0.6 μm to 1 μm, in a range of 0.7 μm to 1 μm, in a range of 0.8 μm to 1 μm or in a range of 0.9 μm to 1 μm. The size of the pore may vary according to the selected type of filter and the specific requirements of the filtration. The filtration unit may further include sealing mechanisms such as O-rings, gaskets, clamps or any other sealing mechanisms capable of preventing leakage and maintaining a sterile environment. The sealing mechanisms of the filtration unit may comprise materials such as silicone, ethylene propylene diene monomer, or polytetrafluoroethylene. The filtration unit may further comprise auxiliary components selected from the group of pumps, pressure sensors, flow meters, valves and means for monitoring the filtration process.

In one aspect of the invention, the filtration unit may be configured to utilize centrifugal force to separate solid-phase particles from liquid phase. This separation process is facilitated by the implementation of centrifugal filters, which may be strategically designed and positioned within the filtration unit.

In one aspect of the invention, the removal of solid residues from the modified protein hydrolysate by the filtration unit may result in a purified protein hydrolysate derived from modified protein hydrolysate. As used herein, the term “purified protein hydrolysate” refers to protein hydrolysate substantially free from solid residues.

The second aspect disclosed herein relates to the use of precipitating agents to generate precipitates of inositol hexaphosphate and its derivatives. Precipitating agents comprise an organic or inorganic compound of 1) suitable cation with high affinity to inositol hexaphosphate and its derivatives—e.g. bivalent cation of calcium, zinc, cobalt, manganese, trivalent cation of iron or any other suitable cation, and 2) an anion corresponding to pKa, which is higher than pKa of inositol hexaphosphate and phosphoric acid—e.g. acetate, carbonate, or hydroxide or any other suitable anion, or any other combination of mentioned cations or anions.

Addition of salts or hydroxides into water results in dissolution of mentioned salts or hydroxides and its dissociation to cations and anions. Cations, especially divalent or multivalent metal cations, interact with negative inositol hexaphosphate and its derivatives by binding to its phosphate groups through ionic interactions. This binding reduces the solubility of inositol hexaphosphate in water, leading to the formation of insoluble metal-phytate complexes resulting in the creation of precipitates.

Addition of salts or hydroxides into hydrolysis tank containing source of protein and water from the loading tank may result in the generation of free metal ions from the before mentioned salts or hydroxides that may form metal-phytate complexes resulting in formation of precipitates in water containing the source of protein.

In one aspect of the invention, the hydrolysis tank may be composed of various materials and specific volumes and may be configured with a stirring unit, water purification unit, loading tank and storage tank.

In another aspect of the invention, the precipitation process may take place in the second hydrolysis tank, wherein the second hydrolysis tank may be connected to the first hydrolysis tank for the source of protein and to the mixing tank.

In one aspect of the invention, the concentration of precipitating agents may differ according to concentration of inositol hexaphosphate contained in the source of protein. The concentration of precipitating agents may be in the range of 150:1 to 1:1 or in the range of 90:1 to 3:1, or in the range of 60:1 to 10:1 expressed as a ratio of the molar concentration of precipitating agents to the molar concentration of inositol hexaphosphate and/or its derivatives in the reaction mixture.

In one aspect of the invention, the use of precipitating agents on protein hydrolysate may result in a modified protein hydrolysate.

In one aspect of the invention, removal of solid residues from protein hydrolysate by filtration unit may be used. In one aspect of the invention, the solid residues may be further used for the production of edible product, wherein the edible product may be used for human or animal consumption.

In one aspect of the invention, the hydrolysis tank may be connected to the filtration unit by pump. This pump may be used for the transfer of protein hydrolysate to the filtration unit.

In one aspect of the invention, the filtration of the solid residues formed by using precipitation may be performed similarly as previously described.

In one aspect of the invention, the filtration of solid residues from the modified protein hydrolysate by the filtration unit may result in a purified protein hydrolysate.

The third aspect disclosed herein relates to the combination of the previously mentioned two aspects: the use of enzymes having phytase activity and precipitating agents. Combining these aspects may significantly decrease the amount of inositol hexaphosphate and its derivatives.

In one aspect of the invention, addition of enzymes having phytase activity in solid or liquid state or their combination thereof into hydrolysis tank containing source of protein may decrease the amount of inositol hexaphosphate and its derivatives. The addition of precipitating agents in the hydrolysis tank after addition of enzymes having phytase activity results in the precipitation of the remaining inositol hexaphosphate and its derivatives. In another aspect of the invention, the enzymes with phytase activity and precipitating agents may be used in one of two orders.

In another aspect of the invention, the combination of treatment by enzymes having phytase activity and precipitation process may take place in a second hydrolysis tank, wherein the second hydrolysis tank may be connected to the hydrolysis tank for the source of protein and to the mixing tank.

Addition of enzymes having phytase activity in solid or liquid state or in their combination thereof into hydrolysis tank containing the source of protein and water source from loading tank leads to cleavage of phosphate ester bonds in the specific pH and temperature.

Addition of precipitating agents into hydrolysis tank containing the source of protein and water source from the loading tank leads to binding of precipitating agents to inositol hexaphosphate and its derivatives causing formation of precipitates.

The first and second aspect may be combined, wherein inositol hexaphosphate and its derivatives may be removed by the first aspect at least 90% and by the second aspect by up to 10%, by the first aspect at least 80% and by the second aspect by up to 20%, by the first aspect at least 70% and by the second aspect by up to 30%, by the first aspect at least 60% and by the second aspect by up to 40%, by the first aspect at least 50% and by the second aspect by up to 50%, by the first aspect at least 40% and by the second aspect by up to 60%, by the first aspect at least 40% and by the second aspect by up to 60%, by the first aspect at least 30% and by the second aspect by up to 70%, by the first aspect at least 20% and by the second aspect by up to 80%, by the first aspect at least 10% and by the second aspect by up to 90%.

In one aspect of the invention, the ratio of precipitating agent to enzymes having phytase activity may be selected according to the amount of inositol hexaphosphate and its derivatives.

In one aspect of the invention, the use of enzymes having phytase activity and precipitating agents on protein hydrolysate may result in a modified protein hydrolysate. The term “modified protein hydrolysate” refers to protein hydrolysate comprising cleaved or precipitated inositol hexaphosphate and its derivatives or their combination thereof.

In one aspect of the invention, solid residues may be removed from protein hydrolysate by filtration unit. In one aspect of the invention, the solid residues may be further used for the production of edible products, wherein the edible product may be used for human or animal consumption.

In one aspect of the invention, the hydrolysis tank may be connected to the filtration unit by pump. This pump may be used for the transfer of protein hydrolysate to the filtration unit.

In one aspect of the invention, the filtration of the solid residues formed by using precipitation may be performed similarly as previously described.

In one aspect of the invention, the filtration of solid residues from the modified protein hydrolysate by the filtration unit may result in a purified protein hydrolysate.

The purified protein hydrolysate is transferred to the mixing tank where the nutritional additives, shear protectants, anti-foaming agents and/or proliferation additives may be loaded from the loading tank 126 in the preparation of culture medium. The term “Purified protein hydrolysate” refers to a hydrolysate that is free from solid residues in a range of 90% to 100%, in a range of 95% to 100%, in a range of 98% to 100%. The term “culture medium” refers to a mix of at least one of purified protein hydrolysate, nutritional additives, shear protectants and/or anti-foaming agents.

The nutritional additives may include saccharides, mineral compounds, vitamins, amino acids, peptides, organic amines, signaling compounds, oligonucleotides, fatty acids, phospholipids, organic micronutrients or any other appropriate nutritional additive according to the selected source of protein, wherein the selected source of protein may contain low concentration of particular amino acids that are present in a low concentration within the source of protein and which are essential for the cell growth and metabolism. Those particular amino acids may be added subsequently as nutritional additives from a loading tank.

In one aspect of the invention, the mixing tank may comprise a main body constructed from at least one material selected from stainless steel, glass-lined steel, titanium, polyethylene, polypropylene, polytetrafluoroethylene or any other suitable materials. The main body may comprise various shapes, such as cylindrical or rectangular or any other suitable geometries. The input of the mixing tank may be configured as a shaft or funnel, wherein the shaft or funnel may be used for loading the nutritional additives. The mixing tank may further comprise a heating system configured to heat the inner environment of the mixing tank. The mixing tank may comprise mixing mechanisms comprising at least one stirrer, paddle or any other instrument capable of mixing the nutritional additives with the purified protein hydrolysate. The sealing mechanisms of the mixing tank may comprise materials such as silicone, ethylene propylene diene monomer, and polytetrafluoroethylene. The mixing tank may be configured to withstand a maximum temperature of at least 100° C. The mixing tank may further comprise auxiliary components selected from the group of pumps, pressure sensors, flow meters, valves and means for monitoring the hydrolysis reaction.

In one aspect of the invention, the loading tank may be configured to provide nutritional additives in dry or liquid form or in the combination thereof, shear protectants, anti-foaming agents into the mixing tank.

In one aspect of the invention, the loading tank for the addition of nutritional additives may be composed of various materials and specific volumes previously mentioned.

The first, the second and the third aspect may be applied to obtain purified protein hydrolysate, wherein each aspect may prevent the formation of precipitates. This ensures that the filters are not blocked by precipitates and that the provided nutrients are more effectively processed by the cells, as opposed to the unusable precipitates.

The first or the third aspect may be applied to obtain released free phosphates from the inositol hexaphosphate and its derivatives and may be used as nutrition for the cells and thus may not be added via loading tank into mixing tank with other nutritional additives.

In one aspect of the invention, the amount of free phosphate provided from the cleaved inositol hexaphosphate and its derivatives is in a range of 50% to 100% of total phosphate ions in the culture medium, in a range of 60% to 100% of total phosphate ions in the culture medium, in a range of 70% to 100% of total phosphate ions in the culture medium, in a range of 80% to 100% of total phosphate ions in the culture medium, in a range of 90% to 100% of total phosphate ions in the culture medium, in a range of 60% to 90% of total phosphate ions in the culture medium, or in a range of 70% to 80% of total phosphate ions in the culture medium.

In one aspect of the invention, the amount of free phosphate provided from the cleaved inositol hexaphosphate and its derivatives is in a range of 50% to 100% of total phosphate ions in the protein hydrolysate, in a range of 60% to 100% of total phosphate ions in the purified protein hydrolysate, in a range of 70% to 100% of total phosphate ions in the purified protein hydrolysate, in a range of 80% to 100% of total phosphate ions in the purified protein hydrolysate, in a range of 90% to 100% of total phosphate ions in the purified protein hydrolysate, in a range of 60% to 90% of total phosphate ions in the purified protein hydrolysate, or in a range of 70% to 80% of total phosphate ions in the purified protein hydrolysate.

In one aspect of the invention, solid residues may be removed from the culture medium by filtration unit. In one aspect of the invention, the solid residues may be further used for the production of edible or food products, wherein the edible or food product may be used for human or animal consumption. In another aspect of the invention, the solid residues may be removed by centrifugation. In another aspect of the invention, centrifugation and filtration may be used in sequence. Preferably, centrifugation comes before filtration to prolong the lifetime of the filter.

In one aspect of the invention, a protein source used for hydrolysis may comprise enzymes from the group of endopeptidases, exopeptidases, phytases, beta-glucanases, lipases, nucleases, or any other suitable enzyme. These enzymes may be released from the protein source contributing to the hydrolysis process. This process, further referred to as autolysis, may allow for hydrolysis with a reduced need for the addition of exogenous enzymes.

In one aspect of the invention, the culture medium may be prepared by using two or more different types of protein sources. When utilizing these two or more different types of protein sources, they may be referred to as “first protein source”, “second protein source”, etc. This process is further referred to as co-hydrolysis.

In one aspect of the invention, co-hydrolysis may utilize at least one protein substrate comprising endogenously produced enzymes from the group of endopeptidases, exopeptidases, phytases, lipases, nucleases, beta-glucanases or any other suitable enzymatic activity. These enzymes may contribute to the hydrolysis of the protein source comprising the enzymes, as well as the hydrolysis of the other protein sources used. As in autolysis, this may allow for a reduced need of exogenously supplied enzymes. By combining a lower-cost protein source having little to no suitable enzymatic activity with another protein source having high suitable enzymatic activity, the nutrient yield relative to cost may be enhanced in comparison to the separate hydrolysis of either individual protein source.

In one aspect of the invention, the exogenously supplied enzymes may comprise proteolytic enzymes, enzymes having phytase activity, or any other enzyme that may catalyze hydrolysis.

The first protein source may comprise a plant-based protein source that is rich in protein and suitable for enzymatic hydrolysis. For example, the first protein source may comprise soy protein isolate, soy protein concentrate, soy flour, soybeans, pea protein, faba bean protein, rice protein, wheat, wheat gluten, and/or duckweed. The present list of examples is not limiting, and any plant-based protein source may be used. The first protein source may further comprise inositol hexaphosphate and/or its derivatives.

The second protein source may comprise at least one protein-rich microbial biomass. The microorganisms may be capable of autolysis by production of different types of enzymes (e.g. endopeptidases, exopeptidases, beta-glucanases, nucleases, lipases) and/or capable of endogenous production of enzymes resulting in breaking-down of the peptide bonds (e.g. peptidases), phosphate ester bonds (e.g. phytases) and/or glycosidic bonds (e.g. beta-glucanase). The second protein source may contribute to the efficiency of the hydrolysis process. The microorganism may be from the group of yeasts, including but not limited to Saccharomyces cerevisiae, Pichia pastoris, Schizosaccharomyces pombe, Candida utilis, Debaryomyces hansenii, or any other appropriate yeast.

The second protein source may be undergoing autolysis, during which the endogenously produced enzymes degrade cellular components, including proteins. The autolysis process may be triggered by pH change, temperature change and/or addition of chemical agents. Endogenously produced enzymes may be released into the environment as a result of cell activity and/or autolysis and/or mechanical cell disruption, allowing the breakdown of plant-based proteins from the first protein source. Additionally, endogenously produced enzymes may catalyze the breakdown of phosphate ester bonds of the inositol hexaphosphate and/or its derivatives into phosphates, which may be utilized by non-human metazoan cells as nutrients. In another aspect of the invention, endogenously produced enzymes may breakdown nucleic acids into free nucleotides and/or nucleosides (including the deoxy variants) and/or free phosphates, which may be utilized by non-human metazoan cells as nutrients (notably as precursors for nucleic acid synthesis). In another aspect of the invention, endogenously produced enzymes may breakdown glycosidic bonds within carbohydrate molecules resulting in release of oligosaccharides, disaccharides, and/or monosaccharides, which may subsequently be utilized as energy sources or structural components by non-human metazoan cells.

In one aspect of the invention, the cells forming the second protein source may be genetically modified and/or otherwise suitably engineered and/or grown under conditions optimized to facilitate an increased production of endogenous enzymes, preferably from the group of endopeptidases, exopeptidases, phytases and/or beta-glucanases. The genetic modification may comprise an insertion of at least one heterologous, or homologous gene encoding an enzyme for breaking down peptide bonds and/or at least one gene encoding an enzyme for breaking down phosphate ester bonds, or a combination thereof. Such modification may further increase the hydrolysis process and may reduce the need to add enzymes during the process.

In another aspect of the invention, the expression of at least one inserted gene encoding an enzyme from the group of exopeptidases, endopeptidases, phytases, or beta-glucanases, or a combination thereof, in the genetically modified cells of the second protein source may be altered by its promoter.

In one aspect of the invention, the endogenously produced enzymes may comprise one or more naturally occurring enzymes from the particular microorganism. These enzymes may comprise, for example, lipase, phosphoesterase, phytase, endopeptidase, exopeptidase, beta-glucanase, nuclease or any other naturally occurring enzyme within the particular microorganism. Additionally, the endogenously produced enzymes, whose genes were incorporated via genetic modification may also comprise enzymes whose sequences were artificially designed and developed through computational methods, including machine learning techniques. These artificially designed enzymes are configured to perform substantially the same function as the naturally occurring enzyme described herein.

The extent of enzyme production may vary depending on the strain used as a second protein source, cultivation conditions, and nutrient availability. In some aspects of the invention, the second protein source may comprise enzymes having endopeptidase activity in the range of 0.0001 to 300,000 TU, or in the range of 0.001 to 30,000 TU, or in the range of 0.01 to 3000 TU (one tyrosine unit is equal to amount of enzyme which liberates one micromole of tyrosine equivalents from casein at 40° C. and pH 7), enzymes having exopeptidase activity in the range of 0.0001 to 200,000 LAPU, or in the range of 0.001 to 20,000 LAPU, or in the range of 0.01-2000 LAPU (one LAPU unit is defined as amount of enzyme which liberates one micromole of p-nitroaniline from L-leucine-p-nitroanilide in one minute at 37° C. and pH 8), enzymes having phytase activity in the range of 0.0001 to 3,000,000 FTU, or in the range of 0.001 to 300,000 FTU or in the range of 0.01 to 30,000 FTU (one phytase unit is equal to amount of enzyme that liberates one micromole of phosphate in one minute from phytate at 37° C. and pH 5.5) and enzymes having beta-glucanase activity in the range of 0.00001 to 10,000 FBG, or in the range of 0.0001 to 1000 FBG, or in the range of 0.001 to 100 FBG (one fungal beta-glucanase unit is defined as amount of enzyme liberating reducing carbohydrates at a rate corresponding to one micromole of glucose per minute from beta-glucan at 37° C. and pH 5) per 1 g of the second protein source. These endogenously produced enzymes contribute to the partial hydrolysis of proteins derived from the first and/or the second protein source. Additionally, these endogenously produced enzymes may contribute to the degradation of inositol hexaphosphate and/or its derivatives, and beta-glucans and/or its derivatives. As a result, the endogenous enzyme production reduces the supplementation of any exogenously supplied enzymes, thereby decreasing the overall requirement for externally supplied enzymatic additives.

In another aspect of the invention, the preparation of the hydrolysis reaction mixture by combination of the first and the second protein source may be carried out in water, optionally supplemented with suitable additives such as acids, bases, salts or buffers. The first protein source and the second protein source may be present in the hydrolysis reaction mixture in the substrate ratios by pure protein content and/or by whole substrate weight. The ratio of the first protein source to the second protein source may be expressed as pure protein content and/or whole substrate content. The specific ratio may vary depending on the desired amino acid profile, enzymatic activity, the combination of protein sources used, and/or other parameters, such as temperature or pH.

The first protein source and the second protein source may be in a ratio of substrates from 0.1:99.9 to 99.9:0.1 by pure protein content, or 0.5:99.5 to 99.5:0.5 by whole substrate weight, ratio of substrates from 1:99 to 99:1 by pure protein content or 2.5:97.5 to 97.5:2.5 by whole substrate weight, ratio of substrates from 2.5:97.5 to 97.5:2.5 by pure protein content or 5:95 to 95:5 by whole substrate weight. In one aspect of the invention, the plant-based protein source (first protein source) may provide a majority of amino acids to the final protein hydrolysate.

In another aspect of the invention, a protein source comprising microbial biomass may be introduced into the hydrolysis reaction while the microbial cells are viable. Viable microbial cells may survive and/or grow in the hydrolysis mixture for some time, until the temperature reaches the deactivation limit. This may allow for a higher amount of endogenous enzymes to be introduced into the hydrolysis mixture. The hydrolysis reaction mixture may be supplemented with nutritional additives to support the growth of the cells from the source of protein comprising microbial biomass.

In one aspect of the invention, to catalyze the breakdown of peptide and phosphate ester bonds from the first and second protein sources, the hydrolysis reaction mixture may be supplemented with exogenous enzymes. The ratio of exogenous enzyme to substrate may be expressed as a ratio of the concentration of enzyme to the concentration of protein in the reaction mixture. The concentration of the enzyme may be determined by the Bradford assay, BCA assay or other protein determination assays. This ratio may vary depending on the type of substrate, its amount, and the intended purpose of hydrolysis. The substrate may comprise, for example, the first and the second protein sources. The enzymes may comprise, for example, at least one endopeptidase, exopeptidase, beta-glucanase, phytase, any appropriate exogenous enzyme, or a combination thereof, with specific ratios optimized according to the particular application. The hydrolysis mixture may comprise, for example, soy protein as the first protein source and P. pastoris as the second protein source. The ratio of exogenous enzyme or enzymes to the relevant substrate may be, for example, within the following ranges (the ratio of the peptidases is expressed to the final substrates mixture, the phytase is expressed to the first substrate and the beta-glucanase is expressed to the second substrate):
a.) the first set of ratios may be 0% to 20% of endopeptidases, 0% to 20% of exopeptidases, 0% to 2% of phytase and/or 0.01% to 20% of beta-glucanase.
b.) the second set of ratios may be 0.05% to 10% of endopeptidases, 0.05% to 10% of exopeptidases, 0.0005% to 1% of phytase and/or 0.05% to 10% of beta-glucanase.
c.) the third set of ratios may be 0.1% to 2% of endopeptidases, 0.1% to 2% of exopeptidases, 0.001% to 0.2% of phytase and/or 0.01% to 5% of beta-glucanase.

The choice of specific enzyme ratios may depend on the relative proportions of the first and second protein sources and their respective concentrations.

In one aspect of the invention, the endogenously produced enzymes and/or exogenously supplied enzymes may catalyze the breakdown of the peptide bonds within the sources of protein resulting in the formation of the protein hydrolysate.

Additionally, the endogenously produced enzymes and/or exogenously supplied enzymes may catalyze the breakdown of the phosphate ester bonds of the inositol hexaphosphate and/or its derivatives within the sources of protein.

Additionally, the endogenously produced enzymes and/or exogenously supplied enzymes may catalyze the breakdown of the glycosidic bonds of the beta-glucan and/or its derivatives within the sources of protein.

In one aspect of the invention, the breakdown of the peptide bonds within the sources of protein, the breakdown of the phosphate ester bonds of the inositol hexaphosphate and/or its derivatives and/or the breakdown of the glycosidic bonds of the beta-glucan or its derivatives may occur simultaneously and may result in formation of modified protein hydrolysate.

The modified protein hydrolysate may be thermally treated at the end of hydrolysis to deactivate enzymes and/or microbial cells. In one aspect of the invention, this treatment may be at a low temperature in the range of 80° C. to 120° C., in the range of 85° C. to 100° C., or in the range of 90° C. to 95° C.

The modified protein hydrolysate may be thermally treated at the end of hydrolysis to deactivate enzymes and/or microbial cells. In one aspect of the invention, this treatment may be at low temperature in the range of 15 minutes to 180 minutes, or in the range of 20 minutes to 120 minutes, or in the range of 25 minutes to 60 minutes.

In another aspect of the invention, the modified protein hydrolysate may be thermally treated at the end of hydrolysis to deactivate enzymes and/or microbial cells. In one aspect of the invention, this treatment may be at high temperature in the range 80° C. to 160° C., or in the range of 100° C. to 155° C., or in the range of 110° C. to 150° C.

In another aspect of the invention, the modified protein hydrolysate may be thermally treated at the end of hydrolysis to deactivate enzymes and/or microbial cells. In one aspect of the invention, this treatment may be at high temperature in the range of 3 seconds to 300 seconds, or in the range of 5 seconds to 60 seconds.

The low temperature method may be performed in a hydrolysis tank, and the low temperature and/or high temperature method may be performed in the hydrolysis tank configured as flash pasteurizer or another suitable continuous flow heating device.

Further, the solid residues from the modified protein hydrolysate may be removed to obtain a purified protein hydrolysate. The purified protein hydrolysate may subsequently be supplemented with nutritional additives to formulate a culture medium suitable for the cultivation of non-human metazoan cells.

In one aspect of the invention, the terms “protein source contributing to the hydrolysis process”, “second protein source” or “source of protein comprising microbial biomass” may be interchangeable.

In one aspect of the invention, a method for co-hydrolyzing the protein source may comprise; providing at least two protein sources to form hydrolysis reaction mixture, wherein a first protein source comprises a plant-based protein source; and a second protein source comprises at least one microorganism; and wherein wherein the second protein source comprises endogenously produced enzymes suitable for co-hydrolysis.

In one aspect of the invention, a method for co-hydrolyzing the protein source may comprise:
i) providing at least two protein sources to form hydrolysis reaction mixture, wherein:
   a) a first protein source comprises a plant-based protein source; and
   b) a second protein source comprises at least one microorganism;
wherein the second protein source comprises endogenously produced enzymes suitable for co-hydrolysis; and
ii) combining the at least two protein sources to form a hydrolysis mixture to initiate the co-hydrolysis,
wherein the endogenously produced enzymes from the second protein source catalyze:
   a) the breakdown of peptide bonds to release peptides and amino acids; and
   b) the breakdown of phosphate ester bonds of inositol hexaphosphate and/or its derivatives to release phosphate ions;

In one aspect of the invention, a method for co-hydrolyzing the protein source may comprise:
i) providing at least two protein sources to form hydrolysis reaction mixture, wherein:
   a) a first protein source comprises a plant-based protein source; and
   b) a second protein source comprises at least one microorganism;
wherein the second protein source comprises endogenously produced enzymes suitable for co-hydrolysis;
and
ii) combining the at least two protein sources to form a hydrolysis mixture to initiate the co-hydrolysis,
wherein the endogenously produced enzymes from the second protein source catalyze:
   a) the breakdown of peptide bonds to release peptides and amino acids; and
   b) the breakdown of phosphate ester bonds of inositol hexaphosphate and/or its derivatives to release phosphate ions;
iii) providing exogenously supplied enzymes for the hydrolysis.

In one aspect of the invention, a method for producing culture medium by co-hydrolysis of a protein source may comprise:
i) providing at least two protein sources, wherein:
   a) a first protein source comprises plant-based protein source; and
   b) a second protein source comprises at least one microorganism;
wherein the hydrolysis mixture is subjected to co-hydrolysis by endogenously produced enzymes from the second protein source; and
ii) providing exogenously supplied enzymes for the hydrolysis;
wherein the exogenously supplied enzymes and endogenously produced enzymes exert a synergic effect in the generation of protein hydrolysate; and
iii) purifying the resulting protein hydrolysate to prepare a culture medium for the cultivation of non-human metazoan cells.

In one aspect of the invention, a culture medium may comprise:
i) a purified protein hydrolysate, obtained by the method comprising at least two protein sources, wherein a first protein source comprises a plant-based protein source and a second protein source comprises microorganisms with endogenously produced enzymes; and
ii) at least one nutritional additive.

In one aspect of the invention, a culture medium may comprise:
i) a purified protein hydrolysate, obtained by the method comprising at least two protein sources, wherein a first protein source comprises a plant-based protein source and a second protein source comprises microorganisms with endogenously produced enzymes; and
ii) at least one nutritional additive,
wherein the culture medium is characterized by enhanced bioavailability of peptides and amino acids and phosphate ions, thereby promoting the proliferation of non-human metazoan cells.

In one aspect of the invention, a cultivation system for the preparation of the culture medium by co-hydrolysis may comprise:
i) at least one culture medium tank, configured for the preparation of culture medium,
wherein the culture medium tank comprises at least one of: a mixing tank, a hydrolysis tank, a storage tank, a loading tank, or a waste medium tank,
wherein the enzymatic co-hydrolysis may be performed in the hydrolysis tank by combination of at least two protein sources, wherein a second protein source comprises at least one microorganism with endogenously produced enzymes and which are combined with exogenously supplied enzymes, thereby resulting in the breakdown of peptide bonds of protein source and breakdown of phosphate ester bonds of inositol hexaphosphate and its derivatives.

In one aspect of the invention, a cultivation system for the preparation of the culture medium by co-hydrolysis may comprise:
i) at least one culture medium tank, configured for the preparation of culture medium,
wherein the culture medium tank comprises at least one of: a mixing tank, a hydrolysis tank, a storage tank, a loading tank, or a waste medium tank,
wherein the enzymatic co-hydrolysis may be performed in the hydrolysis tank by combination of at least two protein sources, wherein a second protein source comprises at least one microorganism with endogenously produced enzymes and which are combined with exogenously supplied enzymes, thereby resulting in the breakdown of peptide bonds of protein source and breakdown of phosphate ester bonds of inositol hexaphosphate and its derivatives; and
ii) a control unit.

In one aspect of the invention, the composition of the culture medium may be defined in terms of the total input of medium components into the cultivation process. In this aspect of the invention, summary amounts of components introduced into the cultivation process at any time point over its entire duration are provided. Furthermore, in this aspect of the invention, the provided concentration ranges for the individual medium components describe the total amount of the given component introduced into the cultivation process at any time point during the cultivation process in relation to the volume of spent culture medium which exits the process. The spent culture medium may exit the cultivation process together with the cultivated cells (harvesting), or separately from the cultivated cells (perfusion). The cultivation process may further have the characteristics of a batch process, where all of the components are introduced into the cultivation process at a single time point and the harvest is performed at a single time point, a fed-batch process, where some components may be introduced after the start of the process and the harvest is done at a single time point, a continuous process, where components may be introduced during the whole duration of cultivation and harvesting may be performed during the whole duration of cultivation, or a combination of the described characteristics. For brevity, this aspect of the invention will be referred to herein as “total input”.

In another aspect of the invention, the composition of the culture medium may be described in terms of the concentration of components which are present at a particular time point during the cell cultivation process in the culture medium. In this aspect of the invention, the provided concentration ranges for the individual medium components describe the concentrations present in the culture medium in the cultivation device at any time point during the cultivation process. For brevity, this aspect of the invention will be referred to herein as “momentary composition”.

The total inputs into the culture medium according to the invention may comprise an optimized ratio of essential amino acids, which may be sourced from a protein hydrolysate, in combination with at least one type of compound selected from a group comprising: saccharides, vitamins and organic micronutrients, mineral compounds, iron supplementation compounds, organic amines, shear protectants, anti-foaming agents or a combination thereof. The media may also contain other compounds, like fatty acids, phospholipids, additional amino acids or oligonucleotides, for example. Media according to the invention with an optimized ratio of amino acids and other nutrients may facilitate efficient production of biomass and a low production of waste metabolites, such as ammonia or lactate, by the cells.

An optimized ratio of essential amino acids is such that essential amino acids may be introduced into the cultivation process in any ratio where the percentage of essential amino acids that can be converted into cellular protein is in the range of 5% to 100%, or in the range of 20% to 90%, or in the range of 30% to 80%. The term “highest possible conversion efficiency” determines what percent of the essential amino acids provided to the cells can be converted into cellular protein, assuming no loss of amino acids to catabolismconversion to other compounds (nucleic acids, for example), or spontaneous degradation.

The highest possible conversion efficiency is determined by the essential amino acid that is the most limiting to the cells. It is calculated such as that for all individual essential amino acids added to the medium in any form at any time point during the cultivation process, the content of that particular essential amino acid in the culture media as a fraction of total essential amino acid content added in any form at any time point to the culture media is divided by the content of that individual amino acid in cellular protein as a fraction of total content of essential amino acids in the lowest obtained ratio, in other words the ratio for the essential amino acid which forms the lowest percentage of the amino acids added to the medium in comparison to the percentage of that particular amino acid in cellular biomass, is then multiplied by 100 to obtain the highest possible conversion efficiency of the provided essential amino acids into cellular protein. All percentages in the calculation of highest possible conversion efficiency are percentages by weight.

The amino acids in the culture media may be present in the form of free amino acids or peptides. Non-essential amino acids are omitted in this calculation, as they can be synthesized by the cells and thus are not limiting in terms of the highest possible conversion efficiency. An example of possible essential amino acid content in cellular protein can be seen in Table 1 below.

The above description may be summarized by the following equation:

$${H}_{EAA} = \frac{\frac{{A}_{EAAM}}{Σ {A}_{EAAM}}}{\frac{{A}_{EAAC}}{Σ {A}_{EAAC}}} * 100 ,$$

where
H_{EAA} is the highest conversion efficiency for a particular amino acid,
A_{EAAM} is the content of that particular essential amino acid in 100 g of protein in culture medium,
ΣA_{EAAM} is the total content of all essential amino acids in 100 g of protein in culture medium,
A_{EAAC} is the content of that particular essential amino acid in 100 g of cellular protein and
ΣA_{EAAC} is the total content of all essential amino acids in 100 g of cellular protein.

An example calculation for the essential amino acid tryptophan would proceed as follows: assuming that the total amount of tryptophan added to the culture media over the period of cultivation was 2 g, and the total amount essential amino acids added to the media over the same time period was 100 g. Table 1 shows that in 100 g of cellular protein, out of 44.7 g of total essential amino acids, 1.6 g are tryptophan.

The calculation:

$${H}_{EAA} = \frac{\frac{2}{100}}{\frac{1.6}{44.7}} * 100 = 55.845 \%$$

shows that the highest conversion efficiency for tryptophan is 55.875%. Now, this process is repeated for each of the nine individual essential amino acids. The lowest of nine numbers obtained is the final highest conversion efficiency.

The amount of essential amino acids that can be converted into cellular protein is determined by how closely the total input of essential amino acids into the cultivation process matches the amino acid composition of cellular protein. Because cells cannot synthesize essential amino acids, the essential amino acid with the lowest relative total input into the cultivation process in comparison to its content in cellular protein will limit maximal cell yield and therefore the maximal percentage of essential amino acids converted to cellular protein (this can be understood as an application of Liebig's law of the minimum).

The conversion efficiency for total essential amino acids may be in the range of 5% to 100%, 20% to 100%, 30% to 100%, or 50% to 100%, calculated by the above mentioned equation.

If the essential amino acid composition of cellular protein according to the example mentioned in Table 1 is used, the resulting total inputs of each essential amino acid given as grams per 100 g of the total input of all essential amino acids may be in the ranges summarized in the Table 2.

The ranges of concentrations of amino acids in grams per 100 g of total essential amino acids introduced into the cultivation process may be according to Table 2, regardless of whether the essential amino acid composition of cellular protein is according to Table 1 or not.

It should be noted that for the purpose of this equation, it is necessary to consistently consider amino acid content either as free amino acids, or as amino acids that are part of a peptide chain (in which case the molecular weight of each amino acid must be considered lower by the weight of one water molecule, to account for the fact that water is a byproduct of a peptide bond formation). In the equation above and Tables 1-3, everything is counted as amino acids that form a peptide chain. Elsewhere in the present document, when amino acid input or concentration is discussed, these are calculated with the molecular weights of free amino acids, and when protein input or concentration is discussed, it is assumed that the amino acids are part of a peptide chain for any calculations.

TABLE 1
Example of possible essential amino
acid content in the cellular protein.

| | content [g/100 g |
|---|---|
| Amino acid | cellular protein] |
| His | 2.7 |
| Ile | 5.1 |
| Leu | 8.9 |
| Lys | 8.2 |
| Met | 2.9 |
| Phe | 4.7 |
| Thr | 4.8 |
| Trp | 1.6 |
| Val | 5.8 |
| Sum | 44.7 |

TABLE 2
Ranges of concentrations of amino acids in grams per 100 grams of total
essential amino acids introduced into the cultivation process.

| Amino acid | range 1 | range 2 | range 3 |
|---|---|---|---|
| His | 0.30 to 6.04 | 1.21 to 5.44 | 1.81 to 4.83 |
| Ile | 0.57 to 11.41 | 2.28 to 10.27 | 3.42 to 9.13 |
| Leu | 1.00 to 19.91 | 3.98 to 17.92 | 5.97 to 15.93 |
| Lys | 0.92 to 18.34 | 3.67 to 16.51 | 5.50 to 14.68 |
| Met | 0.32 to 6.49 | 1.30 to 5.84 | 1.95 to 5.19 |
| Phe | 0.53 to 10.51 | 2.10 to 9.46 | 3.15 to 8.41 |
| Thr | 0.54 to 10.74 | 2.15 to 9.66 | 3.22 to 8.59 |
| Trp | 0.18 to 3.58 | 0.72 to 3.22 | 1.07 to 2.86 |
| Val | 0.65 to 12.98 | 2.60 to 11.68 | 3.89 to 10.38 |

However, the composition of cell biomass is somewhat variable, and therefore the values for each essential amino acid in terms of weight percentage of total essential amino acids used in the media may also be in the ranges summarized in the Table 3.

TABLE 3
Ranges of weight percentage concentration of total essential
amino acids introduced into the cultivation process.

| Amino acid | range 4 | range 5 | range 6 |
|---|---|---|---|
| His | 0.2 to 7.9 | 0.8 to 7.1 | 1.2 to 6.3 |
| Ile | 0.3 to 14.9 | 1.5 to 13.4 | 2.3 to 11.9 |
| Leu | 0.7 to 25.9 | 2.7 to 23.3 | 4.1 to 20.8 |
| Lys | 0.6 to 23.9 | 2.5 to 21.5 | 3.8 to 19.1 |
| Met | 0.2 to 8.5 | 0.9 to 7.6 | 1.3 to 6.8 |
| Phe | 0.3 to 13.7 | 1.4 to 12.3 | 2.2 to 11.0 |
| Thr | 0.3 to 14.0 | 1.5 to 12.6 | 2.2 to 11.2 |
| Trp | 0.1 to 4.7 | 0.5 to 4.2 | 0.7 to 3.8 |
| Val | 0.4 to 16.9 | 1.8 to 15.2 | 2.7 to 13.5 |

Amino acids may be introduced into the cultivation process in the form of free amino acids, salts of amino acids, esters of amino acids, or any other suitable derivatives, as well as oligopeptides, for example dipeptides, tripeptides or tetrapeptides, or polypeptides.

The total input of hydrolysate (expressed as protein dry weight) introduced into the culture medium in the cultivation process may be in the range of 1 g/l to 200 g/l, or in the range of 3 g/l to 100 g/l, or in the range of 10 g/l to 60 g/l, or in the range of 8 g/l to 50 g/l.

The total input of amino acids from hydrolysate, including amino acids in the form of short peptides or suitable bioavailable derivatives, for example phosphoesters, such phosphoserine, or other derivatives, such as methylglycine, is at least 75%, 80%, 85%, 90%, or 95% by weight of the total input of all amino acids into the culture medium.

The culture medium according to the invention may comprise amino acids added separately from the hydrolysate, for example L-methionine, L-cysteine or L-ornithine. The total input of amino acids added separately from hydrolysate may be in the range of 0.02 g/l to 30 g/l, or in the range of 0.05 g/l to 15 g/l, or in the range of 0.1 g/l to 10 g/l.

The total amount of L-cysteine in the culture medium may be in the range of 0.1% to 10%, or 0.5% to 7%, or 1% to 5% by weight with respect to the total amount of hydrolysate protein in the culture medium.

The total amount of L-ornithine in the culture medium is in the range of 0% to 5%, or 0.0001% to 3%, or 0.001% to 0.5% with respect to the total amount of hydrolysate protein in the culture medium.

The total amount of L-methionine in the culture medium may be in the range of 0.05% to 6%, or 0.1% to 3%, or 0.2% to 2% with respect to the total amount of hydrolysate protein in the culture medium.

The total amount of L-tryptophan in the culture medium may be in the range of 0.05% to 6%, or 0.1% to 3%, or 0.2% to 2% with respect to the total amount of hydrolysate protein in the culture medium.

The total amount of L-histidine in the culture medium may be in the range of 0.03% to 4%, or 0.07% to 2%, or 0.15% to 1.5% with respect to the total amount of hydrolysate protein in the culture medium.

The total amount of L-threonine in the culture medium may be in the range of 0.1% to 7%, or 0.2% to 5%, or 0.3% to 3% with respect to the total amount of hydrolysate protein in the culture medium.

The total input of amino acids added to the culture medium separately from the hydrolysate may be in the range of 0.2% to 25%, or in the range of 0.5% to 15%, or in the range of 1% to 10%, expressed as a percentage of the total input of hydrolysate protein into the culture medium.

The culture medium according to the invention may comprise an inorganic source of bioavailable nitrogen, for example ammonia. The total input of inorganic nitrogen source may be in the range 0 g/l to 30 g/l, or in the range 0.5 g/l to 20 g/l, 1 g/l to 10 g/l. The total input of ammonia sourced from hydrolysate may be in the range of 10 mg/l to 1000 mg/l, or in the range of 20 mg/l to 500 mg/l, or in the range of 40 mg/l to 300 mg/l.

As a saccharide may be used at least one compound selected from the group: glucose, fructose, galactose, sucrose, lactose, maltose, or a combination thereof, or any other appropriate saccharide. Total input of saccharides may be in an amount in the range of 1 g/l to 350 g/l, or in the range of 2 g/l to 100 g/l, or in the range of 3 g/l to 20 g/l.

The media may contain at least one of or any combination of the following ions as a mineral compound: Ca²⁺, Cl, Cu²⁺, SO₄²⁻, Fe³⁺, NO³⁻, Fe²⁺, Mg²⁺, K⁺, Na⁺, CO₃²⁻, HCO₃⁻, H₂PO₄⁻, HPO₄²⁻, PO₄³⁻, Zn²⁺, SeO₃²⁻. The media may also contain trace amounts of other mineral compounds and elements, such as cobalt, iodine or manganese.

As the media is prepared by dissolving different constituent compounds in water, any appropriate chemical compound may be used as long as it dissociates to the desired ions in aqueous solution. For example, NaCl and KCl both produce a Cl ion when dissolved. As another example, CuSO₄ and MgCl₂ or MgSO₄ and CuCl₂ may be used to produce Cu²⁺, Mg²⁺, SO₄²⁻ and Cl ions. Assuming equimolar amounts, the resulting aqueous solution will have the same composition for both combinations of compounds used. The total input of mineral compounds introduced into the cultivation process may be in the range of 0.1 g/l to 50 g/l, or in the range of 1 g/l to 20 g/l, or in the range of 3 g/l to 10 g/l.

The total input of Na⁺ may be in the range of 20 mmol/l to 120 mmol/l, or in the range of 30 mmol/l to 100 mmol/l, or in the range of 40 mmol/l to 80 mmol/l.

The total input of Ca²⁺ may be in the range of 0.01 mmol/l to 2 mmol, or in the range of 0.05 mmol/l to 1 mmol, or in the range of 0.1 mmol/l to 0.6 mmol/L.

The total input of Cl⁻ may be in the range of 25 mmol/l to 130 mmol/l, or in the range of 35 mmol/l to 110 mmol/l, or in the range of 45 mmol/l to 90 mmol/l.

The total input of Mg²⁺ may be in the range of 0.3 mmol/l to 10 mmol/l, or in the range of 0.5 mmol/l to 8 mmol/l, or in the range of 1 mmol/l to 5 mmol/l.

The total input of PO₄³⁻ may be in the range of 0.5 mmol/l to 12 mmol/l, or in the range of 0.7 mmol/l to 10 mmol/l, or in the range of 1 mmol/l to 6 mmol/l.

The total input of SO₄²⁻ may be in the range of 0.1 mmol/l to 5 mmol/l, or in the range 0.3 mmol/l to 3 mmol/l, or in the range 0.6 mmol/l to 2 mmol/l.

The total input of K⁺ may be in the range of 2 mmol/l to 18 mmol/l, or in the range of 4 mmol/l to 15 mmol/l, or in the range of 6 mmol/l to 12 mmol/l.

The culture media may contain at least one vitamin of: alpha-tocopherol (vitamin E), ascorbic acid (vitamin C), vitamin B12, biotin, choline, pantothenic acid, folic acid, niacinamide, pyridoxine, riboflavin, thiamine, i-inositol, or a combination thereof. Any appropriate bioactive derivatives or precursors of these compounds may be used. For example, cyanocobalamin may be used instead of vitamin B12, as it can be readily converted to bioactive vitamin B12 by the cells. As another example, thiamine hydrochloride (chloride salt form of thiamine) may be used instead of thiamine. The total input of vitamins introduced into the cultivation process, omitting the vitamins present in lysates or extracts, may be in the range of 0.1 mg/l to 1,000 mg/l, or in the range of 5 mg/l to 500 mg/l, or in the range of 20 mg/l to 300 mg/l.

The total input of choline may be in the range of 10 mg/l to 1,000 mg/l, or in the range of 20 mg/l to 500 mg/l, or in the range of 30 mg/l to 200 mg/l.

The total input of niacinamide (or another vitamer of vitamin B3) may be in the range 3 mg/l to 150 mg/l, or in the range 6 mg/l to 100 mg/l, or in the range of 10 mg/l to 80 mg/l.

As an organic amine may be used at least one compound selected from: putrescine, ethanolamine, or a combination thereof, or any other appropriate amine. Organic amines total input into the cultivation process may be in an amount in the range of 0.01 mg/l to 1,000 mg/l, or in the range of 0.1 mg/l to 100 mg/l, or in the range of 0.5 mg/l to 20 mg/l.

Vitamins and organic amines or their respective precursors or derivatives may be supplied in the form of a lysate or extract, for example autolysed yeast extract or any other appropriate lysate or extract. Extract or lysate for supplementation of micronutrients may be added to the culture media in an amount in the range of 0.01 g/l to 20 g/l, or in the range of 0.1 g/l to 10 g/l, or in the range of 0.5 g/l to 5 g/l.

Iron may be supplemented to the culture medium in compounds with oxidation state iron (III) or iron (II). Iron may be present as free ions, or it may be chelated with a suitable chelating agent to improve its solubility and bioavailability. Chelating agents may include citrate, gluconate, ammonium citrate, EDTA, their combinations, or any other suitable chelating agent. Iron may be introduced into the culture medium bound to the chelating agent (for example, in the form ferric citrate), or iron and the chelating agent may be added separately (for example, in the form of ferric chloride and sodium citrate). The relative amount (w/w) of the total input of the chelating agent to the total input of iron may be in the range of 10000:1 to 1:100, or in the range of 1000:1 to 1:10, or in the range of 10:1 to 1:1. The total input of iron may be in the amount in the range of 0.00001 g/l to 0.5 g/l, or in the range of 0.0001 g/l to 0.1 g/l, or in the range of 0.001 g/l to 0.05 g/l.

The culture medium may comprise a shear protectant to prevent cell damage from mechanical forces caused by mixing and/or sparging in the cultivation device. As a shear protectant may be used at least one of: polyethylene glycol (PEG), methyl cellulose (MC), (hydroxypropyl)methyl cellulose (HPMC), hydroxypropyl cellulose (HPC), carboxymethyl cellulose (CMC), dextran sulfate, or any other appropriate shear protectant, or their combination. Shear protectants may be present in the culture medium in a concentration in the range of 0 g/l to 50 g/l, or in the range of 0.02 g/l to 10 g/l, or in the range of 0.1 g/l to 5 g/l.

As described herein, the physicochemical parameters and composition of the culture medium may be optimized to facilitate fast biomass production, efficient use of nutrients and low production of waste metabolites.

The osmolality of the medium may be in the range of 200 mOsm/kg to 400 mOsm/kg, or range of 250 mOsm/kg to 350 mOsm/kg, or range of 280 mOsm/kg to 330 mOsm/kg. Osmolality may be adjusted before or after the culture medium is introduced into the cultivation device, or a combination of both, and it may be adjusted at a single time point or multiple timepoints. To increase osmolality, NaCl, KCl, glucose, any other appropriate osmolyte or their combination may be used. To decrease osmolality, water or any other appropriate dilute aqueous solution may be used.

The pH of the culture medium in the cultivation device may be in the range of 6 to 8, or in the range of 6.5 to 7.5, or in the range of 6.8 to 7.3. Adjustment of pH may be performed before or after the culture medium is introduced into the cultivation device, or a combination of both, and it may be adjusted at a single time point or multiple timepoints. NaOH, HCL, NaHCO₃, or any other appropriate acid or base may be used to adjust the pH; alternatively, pH may be adjusted by changing the partial pressure of CO₂ in the cultivation device (higher CO₂ partial pressure will result in more CO₂ being dissolved into the culture medium, leading to lower pH). The partial pressure of CO₂ in the cultivation device may be adjusted by changing the percentage of CO₂ in the sparging gas, changing the total pressure in the cultivation device, or changing the mixing and sparging rate in the cultivation device (reducing or increasing CO₂ mass transfer coefficient), or any other appropriate method. The partial pressure of CO₂ in the cultivation device may be in the range of 0.05 kPa to 100 kPa, or in the range 2 kPa to 60 kPa, or in the range 5 kPa to 30 kPa.

The momentary concentration of saccharides in the medium may be in the range 0.005 g/l to 40 g/l, or in the range 0.1 g/l to 20 g/l, or in the range 0.5 g/l to 5 g/l.

The momentary concentration of all amino acids (taking into account both amino acids sourced from the hydrolysate and amino acids added separately and biologically available derivatives, such as esters) and peptides in the medium may be in the range of 0.005 g/l to 30 g/l, or in the range 0.1 g/l to 15 g/l, or in the range 0.5 g/l to 10 g/l.

The composition of culture media as described above may be suitable for cell lines that have been extensively adapted to conditions in vitro. However, some cell types may require additional components in the culture medium, for example protein growth factors, to survive and proliferate. In another aspect of the invention, the culture medium composition suitable for these growth factor dependent cell lines may be described as follows.

The hydrolysates of protein isolates may be used as amino acid sources in culture media according to the invention. Recombinant protein production may be used in culture medium components preparation.

The culture medium according to the invention may comprise macronutrients, micronutrients, signaling compounds and/or other components. The components may be dissolved, for example, in purified water, or in water with inorganic salts, for example phosphate buffer saline (PBS) or water or PBS with Bovine serum albumin (BSA), for example 1% BSA in total.

The signaling compounds may vary according to the specific cell type used in the cultivation in the cultivation device. Examples of those cells may be fibroblasts, myoblasts, adipocytes and their precursors or a combination thereof.

The signaling compounds may or may not induce specific change in the cell fate. Examples of these changes may be stimulation of proliferation and/or stimulation of differentiation. The signaling compounds may be used in a certain order during a certain time period. Examples of those may be the usage of a signaling compound for stimulation of proliferation which is then in the media substituted with the signaling compound for differentiation induction. The precise order of dosing of signaling compounds may or may not be correlated or cross-linked with other tools which affect the cell fate during cultivation.

Signaling compounds for various cell types aimed for stimulation of proliferation may comprise, for example, at least one of the following signaling proteins: FGF family ligands, insulin, insulin like growth factor (IGF) family ligands, TGF family ligands, or transferrin, or any other appropriate signaling compound.

Signaling compounds for various cell types aimed for myogenic differentiation may comprise at least one of FGF, insulin, TGF, transferrin, IGF, epidermal growth factor (EGF), Bone morphogenic protein (BMP), interleukin 6 (IL-6), or IL-13, or any other appropriate signaling compound.

The amino acids and their derivatives that may be supplied to the media are for example: glycine, L-alanine, L-arginine, L-asparagine L-aspartic acid, L-cystine L-glutamic acid, L-glutamine, L-histidine, L-hydroxyproline, L-ornithine, L-citrulline, L-isoleucine, L-leucine, L-lysine, L-methionine, L-phenylalanine, L-proline, L-serine, L-threonine, L-pyroglutamic acid, L-phosphoserine, L-tryptophan, L-tyrosine or L-valine. For the preparation of the culture medium, the given amino acid may be added in the pure form, or as part of a complex mixture of compounds (for example a hydrolysate), or the hydrates or salts (for example hydrochlorides or sodium salts) of amino acids may be used.

In one aspect of the invention, the culture media may comprise protein hydrolysate as a main source of amino acids. The protein hydrolysate may serve as a source of all important amino acids in culture media according to the invention for the purpose of cell cultivation, or some amino acids may be supplied to the media separately, for example L-methionine, which is found in very low concentrations in most scalable sources of protein. Other different individual amino acids may be supplied separately from a different source than a protein hydrolysate.

The processes of cultivation of cells according to the invention may be performed in cultivation systems, equipment or culture vessels well-known and/or commonly used by any person skilled in the art.

The processes of cultivation of cells according to the invention may be performed in a cultivation system. In one aspect of the invention, the cultivation system 1 is as depicted in FIG. 2. The cultivation system 1 may comprise a seeding tank 2, a cultivation device 3, a harvesting device 4, a control unit 5, and sensors and analytical instruments 6 as depicted in FIG. 2. Optionally, the cultivation system 1 may further comprise a device for preparing food products (not depicted in FIG. 2).

The control unit may control and/or regulate every process taking place within the cultivation system. The control unit may be operated using at least one printed circuit board (PCB) and/or microprocessor with software capable of controlling the cultivation device, regardless of the extensions and scale of the system. The control unit may be connected to at least one central data storage. The cultivation system may comprise one or more subcontrol units.

The culture medium may be prepared in a culture medium tank. The culture medium tank may comprise at least one of: mixing tank, hydrolysis tank, storage tank, loading tank or waste medium tank, or any other appropriate device. The media components may be mixed in a mixing tank, which may be made from stainless steel, glass, or any other suitable material. The mixing tank may be equipped with a stirring unit comprising, for example, a shaft with one or more impellers. The mixing tank may be equipped with a heating system. The temperature of the mixing tank may be in the range of 10° C. to 40° C., or in the range of 15° C. to 38° C., or in the range of 18° C. to 35° C. The mixing tank may be connected to one or more storage tanks. The mixing tank may be connected to one or more cultivation devices, formed for example by a bioreactor. The culture medium components may be mixed directly in the cultivation device. The volume of the mixing tank may be in the range of 500 mL to 100 m³, or in the range of 1 L to 10 m³, or in the range of 2 L to 5 m³, or in the range of 500 L to 3 m³. The storage tanks may be made from stainless steel, glass or any other suitable material. The volume of the storage tank may be in the range of 500 mL to 100 m³, or in the range of 1 L to 5 m³, or in the range of 2 L to 3 m³, or in the range of 500 L to 1 m³. The media components may be dosed into the mixing tank through a sterilization filter, or may be sterilized prior to the placement into the mixing tank or may be sterilized in the mixing tank. The mixing tank may be equipped with different types of sensors, such as, for example, thermal sensor, pH probe, conductometer, or any other type of appropriate sensor according to the needs of the process.

In one aspect of the invention, the first protein source, the second protein source, or exogenously supplied enzymes, or a combination thereof, may be introduced into the hydrolysis tank by loading tank. Alternatively, the input of the hydrolysis tank may be configured as a shaft or funnel.

The hydrolysis of the first protein source by using second protein source and exogenously supplied enzymes may be performed within the hydrolysis tank.

The culture media according to present invention may comprise protein hydrolysate as source of amino acids. The process of medium preparation may have the characteristics of a batch process, a continuous process, or a combination thereof.

A more detailed description of the cultivation system, the culture medium, the protein hydrolysate, the cell cultivation methods, the products from the non-human metazoan cells, including specific methodologies, formulations, and system parameters, is available in patent patent application PCT/IB2024/059990, which is hereby incorporated by reference and in its entirety. The present application provides a summary of key aspects relevant to the current invention, while additional details regarding the inventions can be found in the referenced document.

### EXAMPLE

### Example 1: Production of Culture Medium Through Co-Hydrolysis of Soy Protein by Saccharomyces cerevisiae

A mixture was prepared by combining 20 g of soy protein isolate with 700 ml of water. The resulting slurry was mixed to ensure uniform dispersion of the protein in the water. The slurry was then pretreated at 90° C. for 30 minutes to enhance soy protein isolate solubility.

After the pretreatment, the homogenized soy protein slurry was cooled to 42° C. and 40 g of baker yeast (Saccharomyces cerevisiae) was added. The volume of the mixture was then adjusted to 1000 mL of total volume by water. The temperature of 42° C. was maintained for the deactivation of yeast cells while providing optimal conditions for digestion substrate with yeast enzymes. Immediately afterward, 2000 μL of endopeptidase (Alcalase) and 2000 μL of beta-glucanase (Viscozyme) were added to the mixture to facilitate pre-digestion of soy protein isolate and faster degradation of yeast cell structures. The resulting mixture was incubated at 42° C. under continuous agitation at 600 rpm for 18 hours to allow for the digestion with both exogenously added enzymes and endogenously produced enzymes by yeast cells.

Alternatively, yeast cells may be activated prior to the co-hydrolysis process by allowing the pretreated soy protein isolate to cool to 25° C. before adding yeast cells. In this method yeast cells are introduced into a homogenized protein slurry for 20 minutes, followed by increasing the temperature to 42° C. to initiate the digestion process.

At the end of the hydrolysis process, 200 μL of exogenous phytase (Maxamyl) was added to support endogenous phytase activity from S. cerevisiae and thereby enhancing the degradation of the remaining inositol hexaphosphate and its derivatives.

After the hydrolysis process, the mixture was subjected to heat temperature at 90° C. for 30 minutes to inactivate all enzymes present in the mixture. Subsequently, the mixture was then subjected to centrifugation at 2000 G for 10 minutes to separate the solid residues. The supernatant containing the purified protein hydrolysate was further subjected to heat treatment or filtered through 0.2 μm filter, to remove possible contaminants. The supernatant was then stored for further use.

1-3. (canceled)

4. A method for co-hydrolysis of two or more protein sources, the method comprising:
providing at least first and second protein sources to form a hydrolysis reaction mixture, wherein
the first protein source comprises a plant-based protein source; and
the second protein source comprises at least one microorganism; and wherein
the second protein source comprises at least one endogenously produced enzyme suitable for co-hydrolysis of the first protein source and the second protein source.

5. The method according to claim 4, wherein the co-hydrolysis occurs at a temperature in the range of 40° C. to 75° C., and over a period of constant mixing in the range of 30 minutes to 24 hours.

6. The method according to claim 4, wherein the plant-based protein source comprises soy, pea, rice, wheat, wheat gluten, corn, faba beans, alfalfa, hemp, chickpea, potato, pumpkin, rapeseed, red lentil, Spirulina, Chlorella, sunflower, water lentil, mung beans, flax, brewer spent grain, distillers spent grain (DDGS), or tomato pomace.

7. The method according to claim 6, wherein the plant-based protein source is subjected to an initial thermal pretreatment at a temperature in the range of 75° C. to 95° C. for a time in the range of 5 minutes to 120 minutes.

8. The method according to claim 4, wherein the second protein source comprises at least one microorganism selected from Saccaromyces cerevisiae, Pichia pastoris, Schizosaccharomyces pombe, Candida utilis, or Debaryomyces hansenii.

9. The method according to claim 4, wherein the co-hydrolysis of the first and second protein sources results in the breakdown of phosphate ester bonds of inositol hexaphosphate, its derivatives, or both.

10. The method according to claim 4, wherein the at least one endogenously produced enzyme comprises one or more naturally occurring enzymes selected from lipase, phosphoesterase, phytase, endopeptidase, exopeptidase, beta-glucanase, and nuclease.

11. The method according to claim 4, wherein the second protein source comprises at least one enzyme having endopeptidase activity at a concentration in the range of 0.0001 to 300,000 TU per 1 g of the second protein source.

12. The method according to claim 4, wherein the second protein source comprises at least one enzyme having exopeptidase activity at a concentration in the range of 0.0001 to 200,000 LAPU per 1 g of the second protein source.

13. The method according to claim 4, wherein the second protein source comprises at least one enzyme having phytase activity at a concentration in the range of 0.0001 to 3,000,000 FTU per 1 g of the second protein source.

14. The method according to claim 4, wherein the second protein source comprises at least one enzyme having beta-glucanase activity at a concentration in the range of 0.00001 to 10,000 FBG per 1 g of the second protein source.

15. The method according to claim 4, wherein the hydrolysis reaction mixture is supplemented with at least one exogenous proteolytic enzyme and at least one exogenous enzyme having phytase activity.

16. The method according to claim 4, wherein the first protein source and the second protein source are present in a ratio of substrates from 0.1:99.9 to 99.9:0.1 by pure protein content.

17. The method according to claim 4, wherein the first protein source and the second protein source are present in a ratio of substrates from 0.5:99.5 to 99.5:0.5 by whole substrate weight.

18. The method according to claim 15, wherein the endogenously produced enzymes and exogenously supplied enzymes act synergistically to catalyze the breakdown of the peptide bonds within the sources of protein, resulting in the formation of a protein hydrolysate.

19. The method according to claim 15, wherein the endogenously produced enzymes and exogenously supplied enzymes act synergistically to catalyze the breakdown of the phosphate ester bonds of the inositol hexaphosphate or its derivatives, the breakdown of the glycosidic bonds of the beta-glucan or its derivatives within the sources of protein, or a combination thereof, resulting in the formation of a modified protein hydrolysate.

20. The method according to claim 19, wherein the modified protein hydrolysate is subjected to thermal treatment to deactivate enzymes, microbial cells, or a combination thereof, wherein the thermal treatment occurs at a temperature in the range of 80° C. to 160° C. for a time in the range of 3 seconds to 300 seconds.

21. The method according to claim 20, wherein the modified protein hydrolysate is subjected to centrifugation, resulting in a purified protein hydrolysate, wherein the purified protein hydrolysate is free from solid residues in a range of 90% to 100%.

22. The method according to claim 19, wherein the purified protein hydrolysate yields peptides with molecular weights ranging from 6.7 kDa to 17 kDa.

23. The method according to claim 4, wherein the hydrolysis reaction mixture is used to prepare a culture medium for the cultivation of non-human metazoan cells.

24. The method according to claim 23, wherein non-human metazoan cells are used for the production of cultured meat.

25. A composition comprising a protein hydrolysate produced by co-hydrolysis of two or more protein sources, wherein the first protein source comprises a plant-based protein source, and the second protein source comprises at least one microorganism that provides endogenously produced enzymes.

26. The composition according to claim 25, wherein the protein hydrolysate is mixed with at least one nutritional additive, resulting in a culture medium.

27. The composition according to claim 26, comprising amino acids added separately in the range of 0.02 g/l to 30 g/l.

28. The composition according to claim 26, wherein the at least one nutritional additive comprises saccharides, mineral compounds, vitamins, amino acids, peptides, organic amines, signaling compounds, oligonucleotides, fatty acids, phospholipids, or organic micronutrients, and wherein the composition further comprises at least one of shear protectants, or signaling compounds.

29. The composition according to claim 28, comprising at least one shear protectant from the group of PEG, MC, HPMC, HPC, CMC, and dextran sulphate, wherein the shear protectants are present in the composition in a concentration in the range of 0 g/l to 50 g/l.

30. The composition according to claim 28, comprising vitamins and organic amines in an amount in the range of 0.0.1 g/l to 20 g/l.

31. The composition according to claim 28, wherein the signaling compounds comprise at least one of the following signaling proteins: FGF family ligands, insulin, IGF family ligands, TGF family ligands, and/or transferrin.

32. The composition according to claim 26, wherein the composition is a culture medium used for the cultivation of non-human metazoan cells.

33. The composition according to claim 32, wherein the non-human metazoan cells are used for the production of cultured meat.
